(19) United States
(12) Patent Application Publication
(10) Pub. No.: US 2020/0308220 A1
(43) Pub. Date: Oct. 1, 2020

(54) AROMATIC-CATIONIC PEPTIDES CONJUGATED TO ANTIOXIDANTS AND THEIR USE IN TREATING COMPLEX REGIONAL PAIN SYNDROME

(71) Applicants: Peter Schiller, Montreal (CA); Irena Berezowska, Montreal (CA)

(72) Inventors: Peter Schiller, Montreal (CA); Irena Berezowska, Montreal (CA)

(21) Appl. No.: 15/779,593

(22) PCT Filed: Nov. 29, 2016
(86) PCT No.: PCT/IB2016/057191

§ 371 Date: May 29, 2018

Related U.S. Application Data
(60) Provisional application No. 62/261,180, filed on Nov. 30, 2015.

(51) Int. Cl. C07K 5/02; C07K 5/11; A61P 25/02

(52) U.S. Cl. CPC C07K 5/0202; A61K 38/00; A61P 25/02; C07K 5/1019

(57) ABSTRACT

Compositions comprising an antioxidant directed or indirectly conjugated to an aromatic-cationic peptide are provide. Said antioxidants are selected from TEMPO, Tro, PBN, AHDP, DBHP, Caf and Hem and may be conjugated to the aromatic-cationic peptide directly or indirectly via a linker to the N-terminus, C-terminus or a side chain of an amino acid residue of the aromatic-cationic peptide. In some embodiments, the aromatic-cationic peptide is 2′,6′-Dmt-D-Arg-Phe-Lys-NH₂, Phe-D-Arg-Phe-Lys-NH₂ or D-Arg-2′,6′-Dmt-Lys-Phe-NH₂. These conjugates have increased antioxidant activity as compared to the unconjugated aromatic-cationic peptide and have utility in treating complex regional pain syndrome.

### CROSS-REFERENCE TO RELATED APPLICATIONS

This application claims priority to U.S. Provisional Patent Application No. 62/261,180, filed Nov. 30, 2015, the entire contents of which are hereby incorporated by reference in their entirety.

### TECHNICAL FIELD

The present technology relates generally to aromatic-cationic peptide compositions where the aromatic-cationic peptide is conjugated to an antioxidant and their use in the prevention and treatment of complex regional pain syndrome.

### BACKGROUND

The following description is provided to assist the understanding of the reader. None of the information provided or references cited is admitted to be prior art.

Complex regional pain syndrome (CRPS) is a chronic pain condition most often affecting one of the limbs (arms, legs, hands, or feet), usually after an injury or trauma to that limb. CRPS is believed to be caused by damage to, or malfunction of, the peripheral and central nervous systems.

### SUMMARY

The present technology provides compositions and methods useful in the prevention, treatment and/or amelioration of complex regional pain syndrome pain.

In one aspect, the present technology provides compositions comprising an aromatic-cationic peptide of the present technology directly or indirectly conjugated to an antioxidant as well as methods for their use. Such molecules are referred to hereinafter as “peptide conjugates.” At least one antioxidant and at least one aromatic-cationic peptide associate to form a peptide conjugate. The antioxidant and aromatic-cationic peptide can associate by any method known to those in the art. Suitable types of associations involve covalent bond formation. By “directly conjugated” is meant that an atom of the antioxidant is covalently bound to an atom of the aromatic-cationic peptide. In some embodiments, the peptide conjugates have the general structure shown below:
aromatic-cationic peptide-antioxidant

By “indirectly conjugated” is meant that the antioxidant and aromatic-cationic peptide are covalently attached to each other through one or more intermediary atoms, i.e., a linker. In some embodiments, the peptide conjugates have the general structure shown below:
aromatic-cationic peptide-linker-antioxidant

The type of association between the antioxidant and aromatic-cationic peptides typically depends on, for example, functional groups available on the antioxidant and functional groups available on the aromatic-cationic peptide. The peptide conjugate linker may be nonlabile.

In some embodiments, provided herein, is a composition an aromatic-cationic peptide disclosed in Section II conjugated to an antioxidant selected from TEMPO (4-hydroxy-2,2,6,6-tetramethylpiperidine-1-oxyl), Tro (Trolox), PBN (phenyl-N-tert-butylnitrone), AHDP (2-amino-5-hydroxy-4,6-dimethylpyrimidine), DBHP (4-hydroxy-3,5-di-tert-butylphenyl), Caf (caffeic acid), and Hcm (7-hydroxycoumarin)). In some embodiments, the aromatic-cationic peptide is selected from 2′,6′-Dmt-D-Arg-Phe-Lys-NH₂, Phe-D-Arg-Phe-Lys-NH₂, and D-Arg-2′,6′-Dmt-Lys-Phe-NH₂. In some embodiments, the aromatic-cationic peptide comprises H-2′,6′-Dmt-D-Arg-Phe-Lys-NH₂. In some embodiments, the aromatic-cationic peptide comprises H-D-Arg-2′,6′-Dmt-Lys-Phe-NH₂.

In another aspect, the present technology provides a peptide conjugate comprising an antioxidant directly or indirectly conjugated to an aromatic-cationic peptide, wherein the aromatic-cationic peptide is selected from the group consisting of: 2′,6′-Dmt-D-Arg-Phe-Lys-NH₂, Phe-D-Arg-Phe-Lys-NH₂, or D-Arg-2′,6′-Dmt-Lys-Phe-NH₂, or any peptide described in Section II; and wherein the antioxidant is selected from TEMPO, Trolox, PBN, AHDP, DBHP, Caf, and Hcm. In some embodiments, the peptide conjugate comprises an antioxidant conjugated to an aromatic-cationic peptide, wherein the aromatic-cationic peptide is selected from the group consisting of: 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂, a peptide of Tables A-E; and wherein the antioxidant is selected from TEMPO, Tro, PBN, AHDP, DBHP, Caf, and Hcm. In some embodiments, the aromatic-cationic peptide is selected from the group consisting of: 2′,6′-Dmt-D-Arg-Phe-Lys-NH₂, Phe-D-Arg-Phe-Lys-NH₂, and D-Arg-2′,6′-Dmt-Lys-Phe-NH₂. In some embodiments, the aromatic-cationic peptide comprises H-2′,6′-Dmt-D-Arg-Phe-Lys-NH₂. In some embodiments, the aromatic-cationic peptide comprises H-D-Arg-2′,6′-Dmt-Lys-Phe-NH₂. In some embodiments, the peptide conjugate has a structure of Formula G, wherein X=TEMPO, AHDP, Tro or Caf, and n=1-4; Formula H, wherein X=PBN, DBHP, or Hcm; Formula J, wherein X=—CO—NH-(TEMPO), —CO—(PBN), —CO-(AHDP), —CO-(DBHP), —NH-(Tro), —NH—(Caf), or —NH—(Hcm), and n=2-6; Formula K, wherein X=TEMPO, AHDP, Tro or Caf, and n=1-4; Formula L, wherein X=PBN, DBHP, or Hcm; Formula M, wherein X=—CO—NH-(TEMPO), —CO—(PBN), —CO-(AHDP), —CO-(DBHP), —NH(Tro), —NH—(Caf), or —NH-(Hcm), and n=2-6; or Formula N, wherein X=(TEMPO)—NH—CO—(CH₂)ₙ—CO—, Tro or Caf, and n=2-6.

In some embodiments, the antioxidant is directly or indirectly conjugated to the N-terminus or C-terminus of the aromatic-cationic peptide. In some embodiments, the antioxidant is directly or indirectly conjugated to a sidechain of an amino acid residue of the aromatic-cationic peptide. In some embodiments, the antioxidant is covalently bound to the aromatic-cationic peptide through a nitrogen or oxygen atom on the aromatic-cationic peptide.

In some embodiments, In some embodiments, the antioxidant is indirectly conjugated to the aromatic-cationic peptide through a linker. In some embodiments, the linker is covalently bound to the aromatic-cationic peptide through a nitrogen on the aromatic-cationic peptide. In some embodiments, the linker is a C₁-C₁₂ linker and/or comprises one or more groups independently selected from the group consisting of a carbonyl, an amine, and an alkylene group. In some embodiments, the linker is selected from the group consisting of —C(O)—(C₁-C₆ alkylene)-C(O)—, —C(O)—(C₁-C₆ alkylene)-NH—, and —NH—(C₁-C₆ alkylene)-NH—.

In another aspect, the present technology provides methods for delivering one or more peptide conjugates to a cell, the method comprising contacting the cell with one or more peptide conjugates, wherein the peptide conjugates comprises an antioxidant conjugated to an aromatic-cationic peptide, wherein the aromatic-cationic peptide is selected from the group consisting of: 2′,6′-Dmt-D-Arg-Phe-Lys-NH₂, Phe-D-Arg-Phe-Lys-NH₂, or D-Arg-2′,6′-Dmt-Lys-Phe-NH₂, or any peptide described in Section II; and wherein the antioxidant is selected from TEMPO, Trolox, PBN, AHDP, DBHP, Caf, and Hcm. In some embodiments, the antioxidant is selected from PBN, DBHP, Caf, and Hcm. In some embodiments, the antioxidant is indirectly conjugated to the aromatic-cationic peptide by a linker.

In another aspect, the present technology provides methods for treating, ameliorating or preventing complex regional pain syndrome in a subject in need thereof. In some embodiments, the method comprises administering a therapeutically effective amount of one or more peptide conjugates, wherein the peptide conjugates comprise an aromatic-cationic peptide conjugated to an antioxidant described in Section I, to the subject thereby treating, amelioration or preventing complex regional pain syndrome. In some embodiments, the complex regional pain syndrome is complex regional pain syndrome-Type I (CRPS-I).

### BRIEF DESCRIPTION OF THE FIGURES

FIG. 1 is a graph comparing the antioxidant activity of H-Dmt-D-Arg-Phe-Lys(Tro[5])—NH₂ (▪), H-Dmt-D-Arg-Phe-Lys(NH—CH₂-Tro)-NH₂ (S) (▴), H-Dmt-D-Arg-Phe-Lys(NH-TEMPO)-NH₂ (□) (peptide conjugated) and H-Dmt-D-Arg-Phe-Lys-NH₂ ([Dmt¹]DALDA) (●) (an aromatic-cationic peptide) in an assay based on inhibition of linoleic acid peroxidation initiated with 2,2′-azabis(2-amidinopropane) (ABAP). A constant rate of peroxidation is represented by the dashed line.

### DETAILED DESCRIPTION

It is to be appreciated that certain aspects, modes, embodiments, variations and features of the present technology are described below in various levels of detail in order to provide a substantial understanding of the present technology.

The present technology provides compositions comprising an aromatic-cationic peptide of the present technology conjugated to an antioxidant. Such molecules are referred to hereinafter as peptide conjugates.

At least one antioxidant selected from TEMPO, Trolox, PBN, AHDP, DBHP, Caf, and Hcm and at least one aromatic-cationic peptide as described in Section II associate to form a peptide conjugate. The antioxidant and aromatic-cationic peptide can associate by any method known to those in the art. Suitable types of associations involve covalent bond formation.

In some embodiments, the peptide conjugates have the general structure shown below:
aromatic-cationic peptide-antioxidant

In some embodiments, the peptide conjugates have the general structure shown below:
aromatic-cationic peptide-linker-antioxidant

The type of association between the antioxidant and aromatic-cationic peptides typically depends on, for example, functional groups available on the antioxidant and functional groups available on the aromatic-cationic peptide. The peptide conjugate linker may be nonlabile.

While the peptide conjugates described herein can occur and can be used as the neutral (non-salt) peptide conjugate, the description is intended to embrace all salts of the peptide conjugates described herein, as well as methods of using such salts of the peptide conjugates. In one embodiment, the salts of the peptide conjugates comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic peptide conjugate may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic peptide conjugates with amino acids, such as aspartate salts and glutamate salts, can also be prepared. The desired salt of an acidic peptide conjugate can be prepared by methods known to those of skill in the art by treating the compound with a base. Examples of inorganic salts of acid conjugates include, but are not limited to, alkali metal and alkaline earth salts, such as sodium salts, potassium salts, magnesium salts, and calcium salts; ammonium salts; and aluminum salts. Examples of organic salts of acid peptide conjugates include, but are not limited to, procaine, dibenzylamine, N-ethylpiperidine, N,N′-dibenzylethylenediamine, and triethylamine salts. Salts of acidic peptide conjugates with amino acids, such as lysine salts, can also be prepared. The present technology also includes all stereoisomers and geometric isomers of the peptide conjugates, including diastereomers, enantiomers, and cis/trans (E/Z) isomers. The present technology also includes mixtures of stereoisomers and/or geometric isomers in any ratio, including, but not limited to, racemic mixtures.

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like.

As used herein, the term “about” encompasses the range of experimental error that may occur in a measurement and will be clear to the skilled artisan.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the term “amino acid” includes naturally-occurring amino acids and synthetic amino acids, as well as amino acid analogues and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogues refer to compounds that have the same basic chemical structure as a naturally-occurring amino acid, i.e., an α-carbon that is bound to a carboxyl group, an amino group, and an R group, e.g., homoserine, norleucine, methionine sulfoxide, methionine methyl sulfonium. Such analogues have modified R groups (e.g., norleucine) or modified peptide backbones, but retain the same basic chemical structure as a naturally-occurring amino acid. Amino acid mimetics refer to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

As used herein, an “isolated” or “purified” polypeptide or peptide is substantially free of cellular material or other contaminating polypeptides from the cell or tissue source from which the agent is derived, or substantially free from chemical precursors or other chemicals when chemically synthesized. For example, an isolated aromatic-cationic peptide would be free of materials that would interfere with diagnostic or therapeutic uses of the agent. Such interfering materials may include enzymes, hormones and other proteinaceous and nonproteinaceous solutes.

As used herein, the term “non-naturally-occurring” refers to a composition which is not found in this form in nature. A non-naturally-occurring composition can be derived from a naturally-occurring composition, e.g., as non-limiting examples, via purification, isolation, concentration, chemical modification (e.g., addition or removal of a chemical group), and/or, in the case of mixtures, addition or removal of ingredients or compounds. Alternatively, a non-naturally-occurring composition can comprise or be derived from a non-naturally-occurring combination of naturally-occurring compositions. Thus, a non-naturally-occurring composition can comprise a mixture of purified, isolated, modified and/or concentrated naturally-occurring compositions, and/or can comprise a mixture of naturally-occurring compositions in forms, concentrations, ratios and/or levels of purity not found in nature.

As used herein, the term “net charge” refers to the balance of the number of positive charges and the number of negative charges carried by the amino acids present in the aromatic-cationic peptides of the present technology. In this specification, it is understood that net charges are measured at physiological pH. The naturally occurring amino acids that are positively charged at physiological pH include L-lysine, L-arginine, and L-histidine. The naturally occurring amino acids that are negatively charged at physiological pH include L-aspartic acid and L-glutamic acid.

As used herein, “peptide conjugate(s)” refers to an aromatic-cationic peptide, such as, e.g., 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂, or pharmaceutically acceptable salt thereof, conjugated to an antioxidant selected from TEMPO, Trolox, PBN, AHDP, DBHP, Caf, and Hcm. In some embodiments, the antioxidant is selected from PBN, DBHP, Caf, and Hcm.

As used herein, the terms “polypeptide,” “peptide,” and “protein” are used interchangeably herein to mean a polymer comprising two or more amino acids joined to each other by peptide bonds or modified peptide bonds, i.e., peptide isosteres, including, but not limited to, reduced peptide bonds (—CH₂—NH—) and N-methylated peptide bonds (—N(CH₃)—CO—). Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, “prevention” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York), incorporated herein by reference in its entirety for any and all purposes. Amino protecting groups include, but are not limited to, mesitylenesulfonyl (Mts), benzyloxycarbonyl (CBz or Z), t-butyloxycarbonyl (Boc), t-butyldimethylsilyl (TBS or TBDMS), 9-fluorenylmethyloxycarbonyl (Fmoc), acetyl (Ac), trifluoroacetyl, tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α-,α-dimethyldimethoxybenzyloxycarbonyl (DDZ), 5-bromo-7-nitroindolinyl, and the like, as well as phosphoryl protecting groups as exemplified by the following structure:

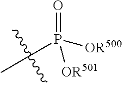

wherein R⁵⁰⁰ and R⁵⁰¹ are each independently hydrogen or a substituted or unsubsituted alkyl, aryl, heterocyclyl, heteroaryl group. Hydroxyl protecting groups include, but are not limited to, Fmoc, TBS, photolabile protecting groups (such as nitroveratryl oxymethyl ether (Nvom)), Mom (methoxy methyl ether), and Mem (methoxyethoxy methyl ether), NPEOC (4-nitrophenethyloxycarbonyl) and NPEOM (4-nitrophenethyloxymethyloxycarbonyl).

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” can be an individual organism, a vertebrate, a mammal, or a human.

As used herein, a “synergistic therapeutic effect” refers to a greater-than-additive therapeutic effect which is produced by a combination of at least two agents, and which exceeds that which would otherwise result from the individual administration of the agents. For example, lower doses of one or more agents may be used in treating a disease or disorder, resulting in increased therapeutic efficacy and decreased side-effects.

As used herein, a “therapeutically effective amount” of a compound refers to compound levels in which the physiological effects of a disease or disorder are, at a minimum, ameliorated. A therapeutically effective amount can be given in one or more administrations. The amount of a compound which constitutes a therapeutically effective amount will vary depending on the compound, the disorder and its severity, and the general health, age, sex, body weight and tolerance to drugs of the subject to be treated, but can be determined routinely by one of ordinary skill in the art.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### I. ANTIOXIDANTS

The antioxidants of the present technology may be selected from TEMPO, Trolox (Tro), PBN, AHDP, DBHP, caffeic acid (Caf), and Hcm. In some embodiments, the antioxidant is selected from PBN, DBHP, Caf, and Hcm.

In some embodiments, TEMPO, Trolox, PBN, AHDP, DBHP, Caf, and Hcm are attached to an aromatic-cationic peptide at a position designated by “--” as indicated below:

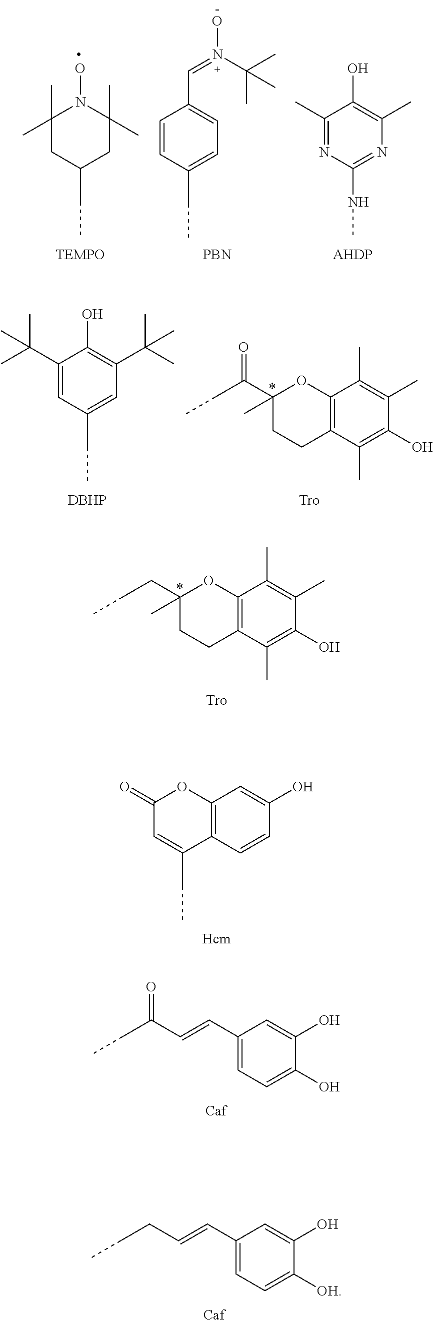

### II. AROMATIC-CATIONIC PEPTIDES AS ACTIVE AGENTS

The aromatic-cationic peptides of the present technology preferably include a minimum of three amino acids, covalently joined by peptide bonds.

The maximum number of amino acids present in the aromatic-cationic peptides of the present technology is about twenty amino acids covalently joined by peptide bonds. In some embodiments, the total number of amino acids is about twelve. In some embodiments, the total number of amino acids is about nine. In some embodiments, the total number of amino acids is about six. In some embodiments, the total number of amino acids is four.

In some aspects, the present technology provides an aromatic-cationic peptide or a pharmaceutically acceptable salt thereof such as acetate salt, tartrate salt, fumarate salt, hydrochloride salt, or trifluoroacetate salt. In some embodiments, the peptide comprises at least one net positive charge; a minimum of three amino acids; a maximum of about twenty amino acids;
a relationship between the minimum number of net positive charges (pₘ) and the total number of amino acid residues (r) wherein 3pₘ is the largest number that is less than or equal to r+1; and

a relationship between the minimum number of aromatic groups (a) and the total number of net positive charges (pₜ) wherein 2a is the largest number that is less than or equal to pₜ+1, except that when a is 1, pₜ may also be 1.

In some embodiments, the peptide is defined by Formula I:

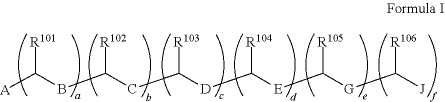

wherein:
one of A and J is

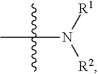

and the other of A and J is

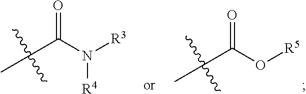

B, C, D, E, and G are each

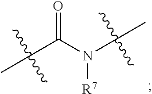

or B, C, D, E, and G are each

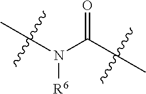

with the proviso that when
   f is 0 and J is not a terminal group, the terminal group is one of G, E, D or C, such that
   one of A and the terminal group is

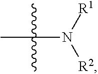

and
the other of A and the terminal group is

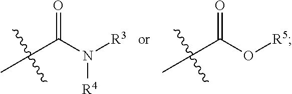

R¹⁰¹ is

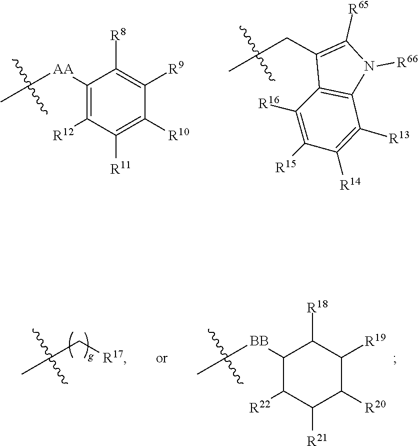

R¹⁰² is

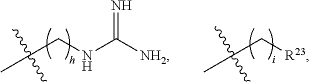

or hydrogen;
R¹⁰³ is

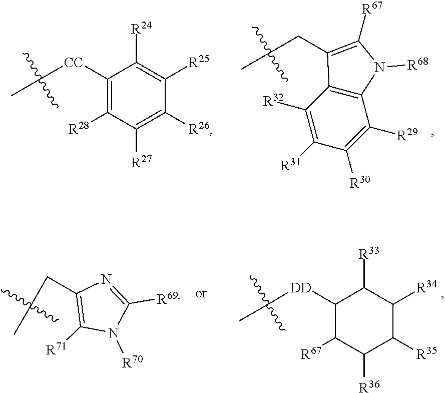

R¹⁰⁴ is

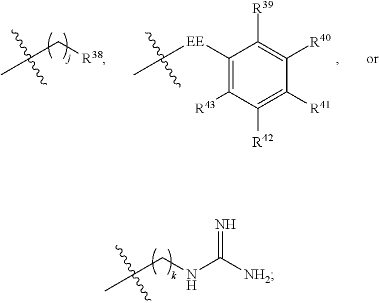

R¹⁰⁵ is

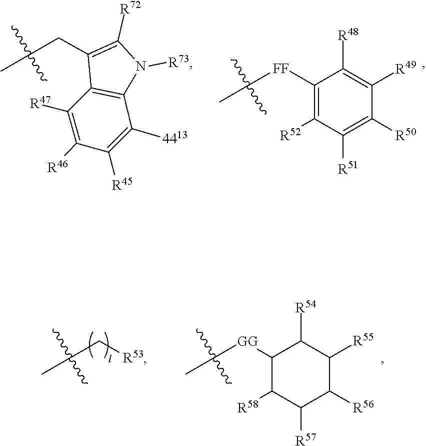

or hydrogen;
R¹⁰⁶ is

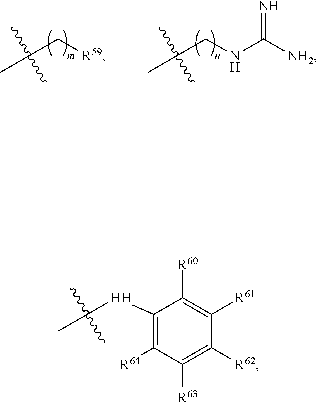

or hydrogen; provided that when R¹⁰², R¹⁰⁴, and R¹⁰⁶ are identical, then R¹⁰¹, R¹⁰³, and R¹⁰⁵ are not identical;
wherein
   R¹, R², R³, R⁴, and R⁵ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R¹ and R² together form a 3, 4, 5, 6, 7, or 8 membered substituted or unsubstituted heterocycyl ring;
   R⁶ and R⁷ at each occurrence are independently a hydrogen or substituted or unsubstituted C₁-C₆ alkyl group;
   R⁸, R⁹, R¹⁰, R¹¹, R¹², R¹³, R¹⁴, R¹⁵, R¹⁶, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵⁴, R⁵⁵, R⁵⁶, R⁵⁷, R⁵⁸, R⁶⁰, R⁶¹, R⁶², R⁶³, R⁶⁴, R⁶⁵, R⁶⁷, R⁶⁹, R⁷¹ and R⁷² are each independently a hydrogen, amino, amido, —NO₂, —CN, —OR^{a}, —SR^{a}, —NR^{a}R^{a}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{a}, C₁-C₄ alkylamino, C₁-C₄ dialkylamino, or perhaloalkyl group;
   R⁶⁶, R⁶⁸, R⁷⁰, and R⁷³ are each independently a hydrogen or substituted or unsubstituted C₁-C₆ alkyl group;
   R¹⁷, R²³, R³⁸, R⁵³, and R⁵⁹ are each independently a hydrogen, —OR^{a}, —SR^{a}, —NR^{a}R^{a}, —NR^{a}R^{b}, —CO₂R^{a}, —(CO)NR^{a}R^{a}, —NR^{a}(CO)R^{a}, —NR^{a}C(NH)NH₂, —NR^{a}-dansyl, or a substituted or unsubstituted alkyl, aryl, or aralkyl group;
   AA, BB, CC, DD, EE, FF, GG, and HH are each independently absent, —NH(CO)—, or —CH₂—;
   R^{a} at each occurrence is independently a hydrogen or a substituted or unsubstituted C₁-C₆ alkyl group;
   R^{b} at each occurrence is independently a C₁-C₆ alkylene-NR^{a}-dansyl or C₁-C₆ alkylene-NR^{a}-anthraniloyl group;
   a, b, c, d, e, and fare each independently 0 or 1,
      with the proviso that a+b+c+d+e+f≥2;
   g, h, k, m, and n are each independently 1, 2, 3, 4, or 5; and
   i, j, and l are each independently 2, 3, 4, or 5;
   provided that
      when i is 4 and R²³ is —SR^{a}, or j is 4 and R³⁸ is —SR^{a}, or l is 4 and R⁵³ is —SR^{a}, then the R^{a} of the —SR^{a} is a substituted or unsubstituted C₁-C₆ alkyl group;
      when J is —NH₂, b and d are 0, a, c, e, f are 1, then R¹⁰³ is

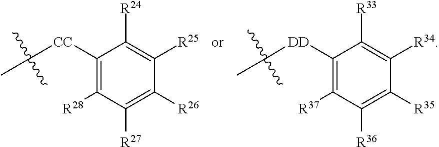

In some embodiments of peptides of Formula I,
R¹, R², R³, R⁴, and R⁵ are each independently a hydrogen or substituted or unsubstituted C₁-C₆ alkyl group;
R⁶ and R⁷ at each occurrence are independently a hydrogen or methyl group;
R⁸, R¹², R¹⁸, R³³, R³⁷, R³⁹, R⁴³, R⁴⁸, R⁵², R⁵⁴, R⁵⁸, R⁶⁰, and R⁶⁴ are each independently a hydrogen or methyl group;
R¹⁰, R²⁰, R²⁶, R³⁵, R⁴¹, R⁵⁰, R⁵⁶, and R⁶² are each independently a hydrogen or —OR^{a};
R⁹, R¹¹, R¹⁹, R²¹, R²⁵, R²⁷, R³⁴, R³⁶, R⁴⁰, R⁴², R⁴⁹, R⁵¹, R⁵⁵, R⁵⁷, R⁶¹, R⁶³, R⁶⁵, R⁶⁶, R⁶⁷, R⁶⁸, R⁶⁹, R⁷⁰, R⁷¹, R⁷², and R⁷³ are each a hydrogen;
R¹⁷, R²³, R³⁸, R⁵³, and R⁵⁹ are each independently a hydrogen, —OH, —SH, —SCH₃, —NH₂, —NHR^{b}, —CO₂H, —(CO)NH₂, —NH(CO)H, or —NH-dansyl group;
AA, BB, CC, DD, EE, FF, GG, and HH are each independently absent or —CH₂—;
R^{a} at each occurrence is independently a hydrogen or a substituted or unsubstituted C₁-C₆ alkyl group;
R^{b} at each occurrence is independently an ethylene-NH-dansyl or ethylene-NH-anthraniloyl group.

In some embodiments of Formula I,

A is

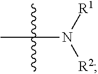

J is

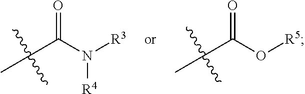

B, C, D, E, and G are each independently

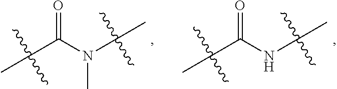

or absent;
with the proviso when f is 0, G is

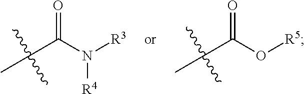

when e and fare 0, E is

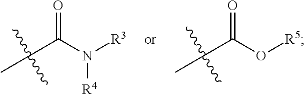

when d, e, and fare 0, D is

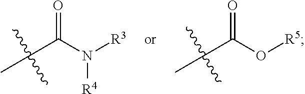

and
when c, d, e, and f are 0, C is

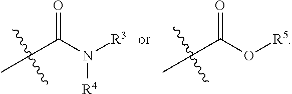

In another embodiment of Formula I,

A is

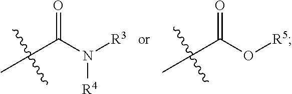

J is

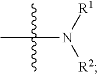

B, C, D, E, and G are each independently

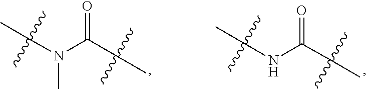

or absent;
with the proviso when f is 0, G is

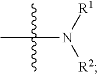

when e and fare 0, E is

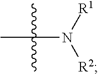

when d, e, and fare 0, D is

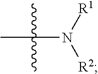

and
when c, d, e, and f are 0, C is

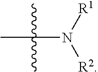

In some embodiments of Formula I, at least one of R¹⁰¹, R¹⁰², R¹⁰⁴, R¹⁰⁵, and R¹⁰⁶ is a basic group, as defined above, and at least one of R¹⁰¹, R¹⁰³, R¹⁰⁴, R¹⁰⁵, and R¹⁰⁶ is a neutral group as defined above. In some such embodiments, the neutral group is an aromatic, heterocyclic or cycloalkyl group as defined above. In some embodiments of Formula I, the peptide contains at least one arginine, such as, but not limited to D-arginine, and at least one 2′,6′-dimethyltyrosine, tyrosine, or phenylalanine. In some embodiments of Formula I, R¹⁰¹ is an alkylguanidinium group.

In some embodiments, the peptide of the present technology is selected from the peptides shown in Tables A or B.

TABLE A
Tyr-D-Arg-Phe-Lys-NH₂
D-Arg-Dmt-Lys-Phe-NH₂
D-Arg-Dmt-Phe-Lys-NH₂
D-Arg-Phe-Lys-Dmt-NH₂
D-Arg-Phe-Dmt-Lys-NH₂
D-Arg-Lys-Dmt-Phe-NH₂
D-Arg-Lys-Phe-Dmt-NH₂
D-Arg-Dmt-Lys-Phe-Cys-NH₂
Phe-Lys-Dmt-D-Arg-NH₂
Phe-Lys-D-Arg-Dmt-NH₂
Phe-D-Arg-Phe-Lys-NH₂
Phe-D-Arg-Phe-Lys-Cys-NH₂
Phe-D-Arg-Phe-Lys-Ser-Cys-NH₂
Phe-D-Arg-Phe-Lys-Gly-Cys-NH₂
Phe-D-Arg-Dmt-Lys-NH₂
Phe-D-Arg-Dmt-Lys-Cys-NH₂
Phe-D-Arg-Dmt-Lys-Ser-Cys-NH₂
Phe-D-Arg-Dmt-Lys-Gly-Cys-NH₂
Phe-D-Arg-Lys-Dmt-NH₂
Phe-Dmt-D-Arg-Lys-NH₂
Phe-Dmt-Lys-D-Arg-NH₂
Lys-Phe-D-Arg-Dmt-NH₂
Lys-Phe-Dmt-D-Arg-NH₂
Lys-Dmt-D-Arg-Phe-NH₂
Lys-Dmt-Phe-D-Arg-NH₂
Lys-D-Arg-Phe-Dmt-NH₂
Lys-D-Arg-Dmt-Phe-NH₂
D-Arg-Dmt-D-Arg-Phe-NH₂
D-Arg-Dmt-D-Arg-Dmt-NH₂
D-Arg-Dmt-D-Arg-Tyr-NH₂
D-Arg-Dmt-D-Arg-Trp-NH₂
Trp-D-Arg-Tyr-Lys-NH₂
Trp-D-Arg-Trp-Lys-NH₂
Trp-D-Arg-Dmt-Lys-NH₂
D-Arg-Trp-Lys-Phe-NH₂
D-Arg-Trp-Phe-Lys-NH₂
D-Arg-Trp-Lys-Dmt-NH₂
D-Arg-Trp-Dmt-Lys-NH₂
D-Arg-Lys-Trp-Phe-NH₂
D-Arg-Lys-Trp-Dmt-NH₂
Cha-D-Arg-Phe-Lys-NH₂
Ala-D-Arg-Phe-Lys-NH₂
2′,6′-Dmp-D-Arg-2′,6′-Dmt-Lys-NH₂
2′,6′-Dmp-D-Arg-Phe-Lys-NH₂
2′,6′-Dmt-D-Arg-Phe-Orn-NH₂
2′,6′-Dmt-D-Arg-Phe-Ahp-NH₂
2′,6′-Dmt-D-Arg-Phe-Lys-NH₂
2′,6′-Dmt-D-Cit-Phe-Lys-NH₂
D-Arg-2′,6′-Dmt-Lys-Phe-NH₂
D-Tyr-Trp-Lys-NH₂
Lys-D-Arg-Tyr-NH₂
Met-Tyr-D-Arg-Phe-Arg-NH₂
Met-Tyr-D-Lys-Phe-Arg
Phe-Arg-D-His-Asp
Phe-D-Arg-2′,6′-Dmt-Lys-NH₂
Phe-D-Arg-His
Trp-D-Lys-Tyr-Arg-NH₂
Tyr-D-Arg-Phe-Lys-Glu-NH₂
Tyr-His-D-Gly-Met
D-Arg-Tyr-Lys-Phe-NH₂
D-Arg-D-Dmt-Lys-Phe-NH₂
D-Arg-Dmt-D-Lys-Phe-NH₂
D-Arg-Dmt-Lys-D-Phe-NH₂
D-Arg-D-Dmt-D-Lys-D-Phe-NH₂
Phe-D-Arg-D-Phe-Lys-NH₂
Phe-D-Arg-Phe-D-Lys-NH₂
D-Phe-D-Arg-D-Phe-D-Lys-NH₂
Lys-D-Phe-Arg-Dmt-NH₂
D-Arg-Arg-Dmt-Phe-NH₂
Dmt-D-Phe-Arg-Lys-NH₂
Phe-D-Dmt-Arg-Lys-NH₂
D-Arg-Dmt-Lys-NH₂
Arg-D-Dmt-Lys-NH₂
D-Arg-Dmt-Phe-NH₂
Arg-D-Dmt-Arg-NH₂
Dmt-D-Arg-NH₂
D-Arg-Dmt-NH₂
D-Dmt-Arg-NH₂
Arg-D-Dmt-NH₂
D-Arg-D-Dmt-NH₂
D-Arg-D-Tyr-Lys-Phe-NH₂
D-Arg-Tyr-D-Lys-Phe-NH₂
D-Arg-Tyr-Lys-D-Phe-NH₂
D-Arg-D-Tyr-D-Lys-D-Phe-NH₂
Lys-D-Phe-Arg-Tyr-NH₂
D-Arg-Arg-Tyr-Phe-NH₂
Tyr-D-Phe-Arg-Lys-NH₂
Phe-D-Tyr-Arg-Lys-NH₂
D-Arg-Tyr-Lys-NH₂
Arg-D-Tyr-Lys-NH₂
D-Arg-Tyr-Phe-NH₂
Arg-D-Tyr-Arg-NH₂
Tyr-D-Arg-NH₂
D-Arg-Tyr-NH₂
D-Tyr-Arg-NH₂
Arg-D-Tyr-NH₂
D-Arg-D-Tyr-NH₂
Dmt-Lys-Phe-NH₂
Lys-Dmt-D-Arg-NH₂
Phe-Lys-Dmt-NH₂
D-Arg-Phe-Lys-NH₂
D-Arg-Cha-Lys-NH₂
D-Arg-Trp-Lys-NH₂
Dmt-Lys-D-Phe-NH₂
Dmt-Lys-NH₂
Lys-Phe-NH₂
D-Arg-Cha-Lys-Cha-NH₂
D-Nle-Dmt-Ahp-Phe-NH₂
D-Nle-Cha-Ahp-Cha-NH₂
D-Arg-Dmt-D-Lys-NH₂
D-Arg-Dmt-D-Lys-Phe-NH₂
Lys-Trp-D-Arg-NH₂
H-Lys-D-Phe-Arg-Dmt-NH₂
H-D-Arg-Lys-Dmt-Phe-NH₂
H-D-Arg-Lys-Phe-Dmt-NH₂
H-D-Arg-Arg-Dmt-Phe-NH₂
H-D-Arg-Dmt-Phe-Lys-NH₂
H-D-Arg-Phe-Dmt-Lys-NH₂
H-Dmt-D-Phe-Arg-Lys-NH₂
H-Phe-D-Dmt-Arg-Lys-NH₂
H-D-Arg-Dmt-Lys-NH₂
H-D-Arg-Dmt-D-Lys-D-Phe-NH₂
H-D-Arg-D-Dmt-Lys-Phe-NH₂
H-D-Arg-Dmt-Phe-NH₂
H-Dmt-D-Arg-NH₂
H-Phe-D-Arg-D-Phe-Lys-NH₂
H-Phe-D-Arg-Phe-D-Lys-NH₂
H-D-Phe-D-Arg-D-Phe-D-Lys-NH₂
H-D-Arg-D-Dmt-D-Lys-D-Phe-NH₂
H-D-Arg-Cha-Lys-NH₂
H-D-Arg-Cha-Lys-Cha-NH₂
H-Arg-D-Dmt-Lys-NH₂
H-Arg-D-Dmt-Arg-NH₂
H-D-Dmt-Arg-NH₂
H-Arg-D-Dmt-NH₂
H-D-Arg-D-Dmt-NH₂
Arg-Arg-Dmt-Phe
Arg-Cha-Lys
Arg-Dmt
Arg-Dmt-Arg
Arg-Dmt-Lys
Arg-Dmt-Lys-Phe
Arg-Dmt-Lys-Phe-Cys
Arg-Dmt-Phe
Arg-Dmt-Phe-Lys
Arg-Lys-Dmt-Phe
Arg-Lys-Phe-Dmt
Arg-Phe-Dmt-Lys
Arg-Phe-Lys
Arg-Trp-Lys
Arg-Tyr-Lys
Arg-Tyr-Lys-Phe
D-Arg-D-Dmt-D-Lys-L-Phe-NH₂
D-Arg-D-Dmt-L-Lys-D-Phe-NH₂
D-Arg-D-Dmt-L-Lys-L-Phe-NH₂
D-Arg-Dmt-D-Lys-NH₂
D-Arg-Dmt-D-Lys-NH₂
D-Arg-Dmt-Lys-Phe-Cys
D-Arg-L-Dmt-D-Lys-D-Phe-NH₂
D-Arg-L-Dmt-D-Lys-L-Phe-NH₂
D-Arg-L-Dmt-L-Lys-D-Phe-NH₂
Dmt-Arg
Dmt-Lys
Dmt-Lys-Phe
Dmt-Phe-Arg-Lys
H-Arg-D-Dmt-Lys-Phe-NH₂
H-Arg-Dmt-Lys-Phe-NH₂
H-D-Arg-2,6-dichloro-L-tyrosine-L-Lys-L-Phe-NH₂
H-D-Arg-2,6-dichlorotyrosine-Lys-Phe-NH₂
H-D-Arg-2,6-difluoro-L-tyrosine-L-Lys-L-Phe-NH₂
H-D-Arg-2,6-difluorotyrosine-Lys-Phe-NH₂
H-D-Arg-2,6-dimethyl-L-phenylalanine-L-Lys-L-Phe-NH₂
H-D-Arg-2,6-dimethylphenylalanine-Lys-Phe-NH₂
H-D-Arg-4-methoxy-2,6-dimethyl-L-phenylalanine-L-Lys-L-
Phe-NH₂
H-D-Arg-4-methoxy-2,6-dimethylphenylalanine-Lys-Phe-NH₂
H-D-Arg-Dmt-Lys-2,6-dimethylphenylalanine-NH₂
H-D-Arg-Dmt-Lys-3-hydroxyphenylalanine-NH₂
H-D-Arg-Dmt-N6-acetyllysine-Phe-NH₂
H-D-Arg-D-Phe-L-Lys-L-Phe-NH₂
H-D-Arg-D-Trp-L-Lys-L-Phe-NH₂
H-D-Arg-D-Tyr-L-Lys-L-Phe-NH₂
H-D-Arg-L-Dmt-L-Lys-2,6-dimethyl-L-phenylalanine-NH₂
H-D-Arg-L-Dmt-L-Lys-3-hydroxy-L-phenylalanine-NH₂
H-D-Arg-L-Dmt-L-Lys-D-Dmt-NH₂
H-D-Arg-L-Dmt-L-Lys-D-Trp-NH₂
H-D-Arg-L-Dmt-L-Lys-D-Tyr-NH₂
H-D-Arg-L-Dmt-L-Lys-L-Dmt-NH₂
H-D-Arg-L-Dmt-L-Lys-L-Trp-NH₂
H-D-Arg-L-Dmt-L-Lys-L-Tyr-NH₂
H-D-Arg-L-Dmt-L-Phe-L-Lys-NH₂
H-D-Arg-L-Dmt-N6-acetyl-L-lysine-L-Phe-NH₂
H-D-Arg-L-Lys-L-Dmt-L-Phe-NH₂
H-D-Arg-L-Lys-L-Phe-L-Dmt-NH₂
H-D-Arg-L-Phe-L-Dmt-L-Lys-NH₂
H-D-Arg-L-Phe-L-Lys-L-Dmt-NH₂
H-D-Arg-L-Phe-L-Lys-L-Phe-NH₂
H-D-Arg-L-Trp-L-Lys-L-Phe-NH₂
H-D-Arg-L-Tyr-L-Lys-L-Phe-NH₂
H-D-Arg-Phe-Lys-Dmt-NH₂
H-D-Arg-Tyr-Lys-Phe-NH₂
H-D-His-L-Dmt-L-Lys-L-Phe-NH₂
H-D-Lys-L-Dmt-L-Lys-L-Phe-NH₂
H-Dmt-D-Arg-Lys-Phe-NH₂
H-Dmt-D-Arg-Phe-Lys-NH₂
H-Dmt-Lys-D-Arg-Phe-NH₂
H-Dmt-Lys-Phe-D-Arg-NH₂
H-Dmt-Phe-D-Arg-Lys-NH₂
H-Dmt-Phe-Lys-D-Arg-NH₂
H-L-Dmt-D-Arg-L-Lys-L-Phe-NH₂
H-L-Dmt-D-Arg-L-Phe-L-Lys-NH₂
H-L-Dmt-L-Lys-D-Arg-L-Phe-NH₂
H-L-Dmt-L-Lys-L-Phe-D-Arg-NH₂
H-L-Dmt-L-Phe-D-Arg-L-Lys-NH₂
H-L-Dmt-L-Phe-L-Lys-D-Arg-NH₂
H-L-His-L-Dmt-L-Lys-L-Phe-NH₂
H-L-Lys-D-Arg-L-Dmt-L-Phe-NH₂
H-L-Lys-D-Arg-L-Phe-L-Dmt-NH₂
H-L-Lys-L-Dmt-D-Arg-L-Phe-NH₂
H-L-Lys-L-Dmt-L-Lys-L-Phe-NH₂
H-L-Lys-L-Dmt-L-Phe-D-Arg-NH₂
H-L-Lys-L-Phe-D-Arg-L-Dmt-NH₂
H-L-Lys-L-Phe-L-Dmt-D-Arg-NH₂
H-L-Phe-D-Arg-L-Dmt-L-Lys-NH₂
H-L-Phe-D-Arg-L-Lys-L-Dmt-NH₂
H-L-Phe-L-Dmt-D-Arg-L-Lys-NH₂
H-L-Phe-L-Dmt-L-Lys-D-Arg-NH₂
H-L-Phe-L-Lys-D-Arg-L-Dmt-NH₂
H-L-Phe-L-Lys-L-Dmt-D-Arg-NH₂
H-Lys-D-Arg-Dmt-Phe-NH₂
H-Lys-D-Arg-Phe-Dmt-NH₂
H-Lys-Dmt-D-Arg-Phe-NH₂
H-Lys-Dmt-Phe-D-Arg-NH₂
H-Lys-Phe-D-Arg-Dmt-NH₂
H-Lys-Phe-Dmt-D-Arg-NH₂
H-Phe-Arg-Phe-Lys-NH₂
H-Phe-D-Arg-Dmt-Lys-NH₂
H-Phe-D-Arg-Lys-Dmt-NH₂
H-Phe-Dmt-D-Arg-Lys-NH₂
H-Phe-Dmt-Lys-D-Arg-NH₂
H-Phe-Lys-D-Arg-Dmt-NH₂
H-Phe-Lys-Dmt-D-Arg-NH₂
L-Arg-D-Dmt-D-Lys-D-Phe-NH₂
L-Arg-D-Dmt-D-Lys-L-Phe-NH₂
L-Arg-D-Dmt-L-Lys-D-Phe-NH₂
L-Arg-D-Dmt-L-Lys-L-Phe-NH₂
L-Arg-L-Dmt-D-Lys-D-Phe-NH₂
L-Arg-L-Dmt-D-Lys-L-Phe-NH₂
L-Arg-L-Dmt-L-Lys-D-Phe-NH₂
L-Arg-L-Dmt-L-Lys-L-Phe-NH₂
Lys-Dmt-Arg
Lys-Phe
Lys-Phe-Arg-Dmt
Lys-Trp-Arg
Phe-Arg-Dmt-Lys
Phe-Arg-Phe-Lys
Phe-Dmt-Arg-Lys
Phe-Lys-Dmt
Arg-Dmt-Lys-Phe-NH₂
Phe-Dmt-Arg-Lys-NH₂
Phe-Lys-Dmt-Arg-NH₂
Dmt-Arg-Lys-Phe-NH₂
Lys-Dmt-Arg-Phe-NH₂
Phe-Dmt-Lys-Arg-NH₂
Arg-Lys-Dmt-Phe-NH₂
Arg-Dmt-Phe-Lys-NH₂
D-Arg-Dmt-Lys-Phe-NH₂
Dmt-D-Arg-Phe-Lys-NH₂
H-Phe-D-Arg-Phe-Lys-Cys-NH₂
D-Arg-Dmt-Lys-Trp-NH₂
D-Arg-Trp-Lys-Trp-NH₂
H-D-Arg-Dmt-Lys-Phe(NMe)-NH₂
H-D-Arg-Dmt-Lys(N^{α}Me)-Phe(NMe)-NH₂
H-D-Arg(N^{α}Me)-Dmt(NMe)-Lys(N^{α}Me)-Phe(NMe)-NH₂
D-Arg-2′6′Dmt-Lys-Phe-NH₂
H-Phe-D-Arg-Phe-Lys-Cys-NH₂
D-Arg-Dmt-Lys-Phe-Ser-Cys-NH₂
D-Arg-Dmt-Lys-Phe-Gly-Cys-NH₂
Gly-D-Phe-Lys-His-D-Arg-Tyr-NH₂
D-Arg-Dmt-Lys-Phe-Met-NH₂
D-Arg-Dmt-Lys-Phe-Lys-Trp-NH₂
D-Arg-Dmt-Lys-Dmt-Lys-Trp-NH₂
D-Arg-Dmt-Lys-Phe-Lys-Met-NH₂
D-Arg-Dmt-Lys-Dmt-Lys-Met-NH₂
H-D-Arg-Dmt-Lys-OH
H-D-Arg-Dmt-OH
H-D-Arg-Dmt-Lys-Phe-OH

TABLE B

| Amino Acid | Amino Acid | Amino Acid | Amino Acid | C-Terminal |
|---|---|---|---|---|
| Position 1 | Position 2 | Position 3 | Position 4 | Modification |
| Tyr | D-Arg | Phe | Orn | NH₂ |
| Tyr | D-Arg | Phe | Dab | NH₂ |
| Tyr | D-Arg | Phe | Dap | NH₂ |
| 2′6′Dmt | D-Arg | Phe | Lys-NH(CH₂)₂- | NH₂ |
| | | | NH-dns | |
| 2′6′Dmt | D-Arg | Phe | Lys-NH(CH₂)₂- | NH₂ |
| | | | NH-atn | |
| 2′6′Dmt | D-Arg | Phe | dnsLys | NH₂ |
| 2′6′Dmt | D-Cit | Phe | Ahp | NH₂ |
| 2′6′Dmt | D-Arg | Phe | Dab | NH₂ |
| 2′6′Dmt | D-Arg | Phe | Dap | NH₂ |
| 3′5′Dmt | D-Arg | Phe | Lys | NH₂ |
| 3′5′Dmt | D-Arg | Phe | Orn | NH₂ |
| 3′5′Dmt | D-Arg | Phe | Dab | NH₂ |
| 3′5′Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′6′Dmt | D-Arg | Tyr | Lys | NH₂ |
| 2′6′Dmt | D-Arg | Tyr | Orn | NH₂ |
| 2′6′Dmt | D-Arg | Tyr | Dab | NH₂ |
| 2′6′Dmt | D-Arg | Tyr | Dap | NH₂ |
| 2′6′Dmt | D-Arg | 2′6′Dmt | Lys | NH₂ |
| 2′6′Dmt | D-Arg | 2′6′Dmt | Orn | NH₂ |
| 2′6′Dmt | D-Arg | 2′6′Dmt | Dab | NH₂ |
| 2′6′Dmt | D-Arg | 2′6′Dmt | Dap | NH₂ |
| 3′5′Dmt | D-Arg | 3′5′Dmt | Arg | NH₂ |
| 3′5′Dmt | D-Arg | 3′5′Dmt | Lys | NH₂ |
| 3′5′Dmt | D-Arg | 3′5′Dmt | Orn | NH₂ |
| 3′5′Dmt | D-Arg | 3′5′Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′6′Dmt | D-Lys | Phe | Dab | NH₂ |
| 2′6′Dmt | D-Lys | Phe | Dap | NH₂ |
| 2′6′Dmt | D-Lys | Phe | Arg | NH₂ |
| 2′6′Dmt | D-Lys | Phe | Lys | NH₂ |
| 3′5′Dmt | D-Lys | Phe | Orn | NH₂ |
| 3′5′Dmt | D-Lys | Phe | Dab | NH₂ |
| 3′5′Dmt | D-Lys | Phe | Dap | NH₂ |
| 3′5′Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′6′Dmt | D-Lys | Tyr | Lys | NH₂ |
| 2′6′Dmt | D-Lys | Tyr | Orn | NH₂ |
| 2′6′Dmt | D-Lys | Tyr | Dab | NH₂ |
| 2′6′Dmt | D-Lys | Tyr | Dap | NH₂ |
| 2′6′Dmt | D-Lys | 2′6′Dmt | Lys | NH₂ |
| 2′6′Dmt | D-Lys | 2′6′Dmt | Orn | NH₂ |
| 2′6′Dmt | D-Lys | 2′6′Dmt | Dab | NH₂ |
| 2′6′Dmt | D-Lys | 2′6′Dmt | Dap | NH₂ |
| 3′5′Dmt | D-Lys | 3′5′Dmt | Lys | NH₂ |
| 3′5′Dmt | D-Lys | 3′5′Dmt | Orn | NH₂ |
| 3′5′Dmt | D-Lys | 3′5′Dmt | Dab | NH₂ |
| 3′5′Dmt | D-Lys | 3′5′Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′6′Dmt | D-Arg | Phe | Arg | NH₂ |
| 2′6′Dmt | D-Lys | Phe | Arg | NH₂ |
| 2′6′Dmt | D-Orn | Phe | Arg | NH₂ |
| 2′6′Dmt | D-Dab | Phe | Arg | NH₂ |
| 3′5′Dmt | D-Dap | Phe | Arg | NH₂ |
| 3′5′Dmt | D-Arg | Phe | Arg | NH₂ |
| 3′5′Dmt | D-Lys | Phe | Arg | NH₂ |
| 3′5′Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′6′Dmt | D-Arg | 2′6′Dmt | Arg | NH₂ |
| 2′6′Dmt | D-Lys | 2′6′Dmt | Arg | NH₂ |
| 2′6′Dmt | D-Orn | 2′6′Dmt | Arg | NH₂ |
| 2′6′Dmt | D-Dab | 2′6′Dmt | Arg | NH₂ |
| 3′5′Dmt | D-Dap | 3′5′Dmt | Arg | NH₂ |
| 3′5′Dmt | D-Arg | 3′5′Dmt | Arg | NH₂ |
| 3′5′Dmt | D-Lys | 3′5′Dmt | Arg | NH₂ |
| 3′5′Dmt | D-Orn | 3′5′Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |
| Trp | D-Arg | Phe | Lys | NH₂ |

2′-methyltyrosine (Mmt); Dimethyltyrosine (Dmt); 2′,6′-dimethyltyrosine (2′6′-Dmt); 3′,5′-dimethyltyrosine (3′5′Dmt); N,2′,6′-trimethyltyrosine (Tmt); 2′-hydroxy-6′-methyltyrosine (Hmt); 2′-methylphenylalanine (Mmp); dimethylphenylalanine (Dmp) 2′,6′-dimethylphenylalanine (2′,6′-Dmp); N,2′,6′-trimethylphenylalanine (Tmp); 2′-hydroxy-6′-methylphenylalanine (Hmp); cyclohexylalanine (Cha); diaminobutyric (Dab); diaminopropionic acid (Dap); β-dansyl-L-α,β-diaminopropionic acid (dnsDap); β-anthraniloyl-L-α,β-diaminopropionic acid (atnDap); biotin (bio); norleucine (Nle); 2-aminohepantoic acid (Ahp); β-(6′-dimethylamino-2′-naphthoyl)alanine (Ald); Sarcosine (Sar)

In another embodiment, the peptide is defined by Formula II:

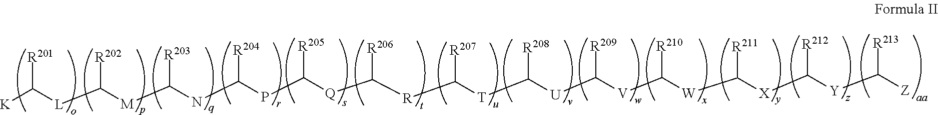

wherein:
one of K and Z is

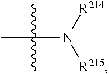

and the other of K and Z is

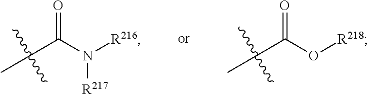

L, M, N, P, Q, R, T, U, V, W, X, and Y are each

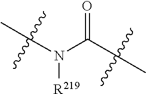

or L, M, N, P, Q, R, T, U, V, W, X, and Y are each

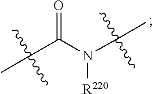

with the proviso that when
   aa is 0 and Z is not a terminal group, the terminal group is one of L, M, N, P, Q, R, T, U, V, W, X, or Y, such that one of K and the terminal group is

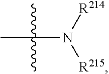

and the other of K and the terminal group is selected from

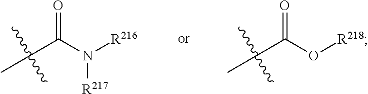

R²⁰¹ is

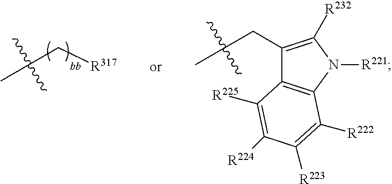

R²⁰² is

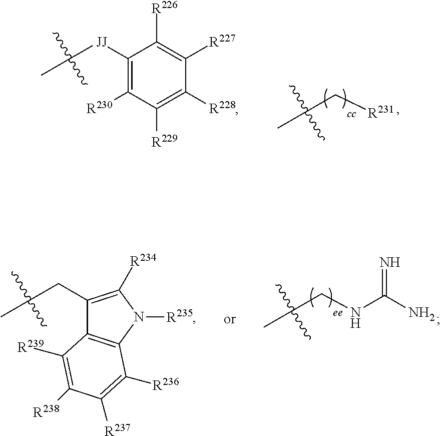

R²⁰³ is

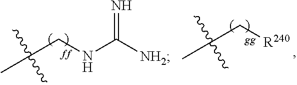

or hydrogen;
R²⁰⁴ is

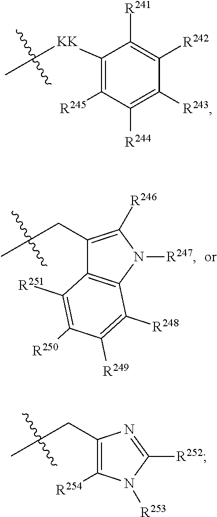

R²⁰⁵ is

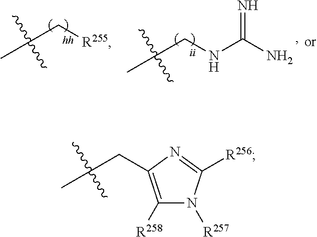

R²⁰⁶ is

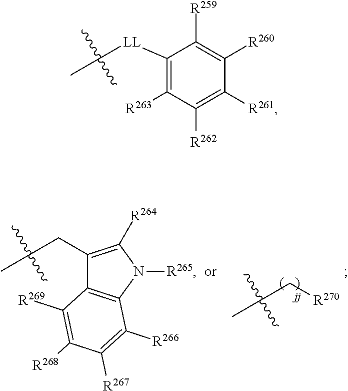

R²⁰⁷ is

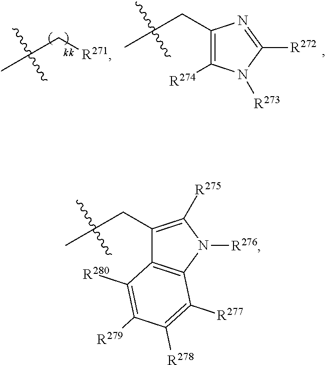

or hydrogen;
R²⁰⁸ is

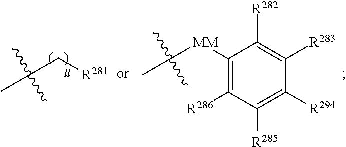

R²⁰⁹ is

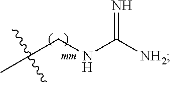

R²¹⁰ is

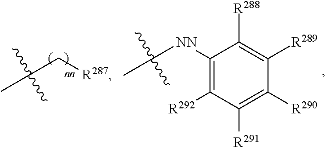

or hydrogen;
R²¹¹ is

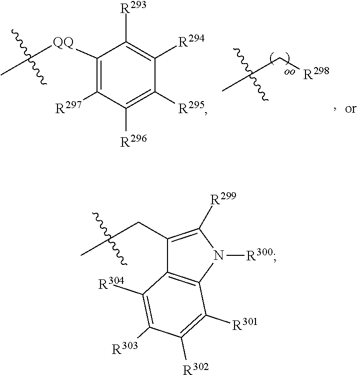

R²¹² is

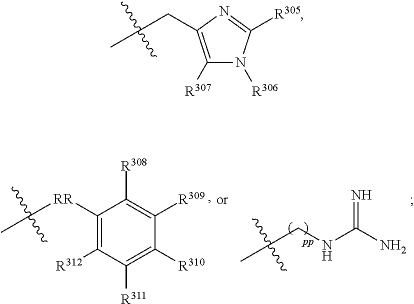

R²¹³ is

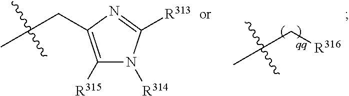

wherein
   R²¹⁴, R²¹⁵, R²¹⁶, R²¹⁷, and R²¹⁸ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R²¹⁴ and R²¹⁵ together form a 3, 4, 5, 6, 7, or 8 membered substituted or unsubstituted heterocycyl ring;
   R²¹⁹ and R²²⁰ are, at each occurrence, independently a hydrogen or substituted or unsubstituted C₁-C₆ alkyl group;
   R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, amino, amido, —NO₂, —CN, —OR^{c}, —SR^{c}, —NR^{c}R^{c}, —F, —Cl, —Br, —I, or a substituted or unsubstituted C₁-C₆ alkyl, C₁-C₆ alkoxy, —C(O)-alkyl, —C(O)-aryl, —C(O)— aralkyl, —C(O)₂R^{c}, C₁-C₄ alkylamino, C₁-C₄ dialkylamino, or perhaloalkyl group;
   R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;
   R²³¹, R²⁴⁰, R²⁵⁵, R²⁷⁰, R²⁷¹, R²⁸¹, R²⁸⁷, R²⁹⁸, R³¹⁶, and R³¹⁷ are each independently a hydrogen, —OR^{c}, —SR^{c}, —NR^{c}R^{c}, NR^{c}R^{d}, CO₂R^{c}, —(CO)NR^{c}R^{c}, —NR^{c}(CO)R^{c}, —NR^{c}C(NH)NH₂, —NR^{c}-dansyl, or a substituted or unsubstituted alkyl, aryl, or aralkyl group;
   JJ, KK, LL, MM, NN, QQ, and RR are each independently absent, —NH(CO)—, or —CH₂—;
   R^{c} at each occurrence is independently a hydrogen or a substituted or unsubstituted C₁-C₆ alkyl group;
   R^{d} at each occurrence is independently a C₁-C₆ alkylene-NR^{c}-dansyl or C₁-C₆ alkylene-NR^{c}-anthraniloyl group;
   o, p, q, r, s, t, u, v, w, x, y, z, and aa are each independently 0 or 1, with the proviso that o+p+q+r+s+t+u+v+w+x+y+z+aa equals 6, 7, 8, 9, 10, or 11;
   cc is 0, 1, 2, 3, 4, or 5; and
   bb, cc, ee, ff, gg, hh, ii, jj, kk, ll, mm, nn, oo, pp, and qq are each independently 1, 2, 3, 4, or 5.

In some embodiments of peptides of Formula II,
R²¹⁴, R²¹⁵, R²¹⁶, R²¹⁷, and R²¹⁸ are each independently a hydrogen or substituted or unsubstituted C₁-C₆ alkyl group;
R²¹⁹ and R²²⁰ are, at each occurrence, independently a hydrogen or methyl group;
R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, methyl, or —OR^{c} group;
R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;
R²³¹ is —(CO)NR^{c}R^{c}, —OW, or a C₁-C₆ alkyl group, optionally substituted with a hydroxyl or methyl group;
R²⁴⁰ and R²⁵⁵ are each independently —CO₂R^{c} or —NR^{c}R^{c};
R²⁷⁰ and R²⁷¹ are each independently —CO₂R^{c};
R²⁸¹ is —SR^{c} or —NR^{c}R^{c};
R²⁸⁷—(CO)NR^{c}R^{c} or —OR^{c};
R²⁹⁸—NR^{c}R^{c}, —CO₂R^{c}, or —SR^{c};
R³¹⁶ is —NR^{c}R^{c};
R³¹⁷ is hydrogen or —NR^{c}R^{c};
JJ, KK, LL, MM, NN, QQ, and RR are each independently absent or —CH₂—;
R^{c} at each occurrence is independently a hydrogen or a substituted or unsubstituted C₁-C₆ alkyl group;
R^{d} at each occurrence is independently a C₁-C₆ alkylene-NR^{c}-dansyl or C₁-C₆ alkylene-NR^{c}-anthraniloyl group;
o, p, q, r, s, t, u, v, w, x, y, z, and aa are each independently 0 or 1,
   with the proviso that o+p+q+r+s+t+u+v+w+x+y+z+aa equals 6, 7, 8, 9, 10, or 11;
cc is 0, 1, 2, 3, 4, or 5; and
bb, cc, dd, ee, ff, gg, hh, ii, jj, kk, 11, mm, nn, oo, pp, and qq are each independently 1, 2, 3, 4, or 5.

In some embodiments of peptides of Formula II,
R²²¹, R²²², R²²³, R²²⁴, R²²⁵, R²²⁶, R²²⁷, R²²⁸, R²²⁹, R²³⁰, R²³², R²³⁴, R²³⁵, R²³⁶, R²³⁷, R²³⁸, R²³⁹, R²⁴², R²⁴⁴, R²⁴⁶, R²⁴⁷, R²⁴⁸, R²⁴⁹, R²⁵⁰, R²⁵¹, R²⁵², R²⁵³, R²⁵⁴, R²⁵⁶, R²⁵⁷, R²⁵⁸, R²⁵⁹, R²⁶⁰, R²⁶², R²⁶³, R²⁶⁴, R²⁶⁵, R²⁶⁶, R²⁶⁷, R²⁶⁸, R²⁶⁹, R²⁷², R²⁷³, R²⁷⁴, R²⁷⁵, R²⁷⁶, R²⁷⁷, R²⁷⁸, R²⁷⁹, R²⁸⁰, R²⁸², R²⁸³, R²⁸⁵, R²⁸⁶, R²⁸⁸, R²⁸⁹, R²⁹¹, R²⁹², R²⁹³, R²⁹⁴, R²⁹⁶, R²⁹⁷, R²⁹⁹, R³⁰⁰, R³⁰¹, R³⁰², R³⁰³, R³⁰⁴, R³⁰⁵, R³⁰⁶, R³⁰⁷, R³⁰⁸, R³⁰⁹, R³¹¹, R³¹², R³¹³, R³¹⁴, and R³¹⁵ are each hydrogen;
R²⁴¹ and R²⁴⁵ are each independently a hydrogen or methyl group;
R²⁴³, R²⁶¹, R²⁸⁴, R²⁹⁰, R²⁹⁵, R³¹⁰ are each independently a hydrogen or OH;
R²³¹ is —(CO)NH₂, an ethyl group substituted with a hydroxyl group, or an isopropyl group;
R²⁴⁰ and R²⁵⁵ are each independently —CO₂H or —NH₂;
R²⁷⁰ and R²⁷¹ are each independently —CO₂H;
R²⁸¹ is —SH or —NH₂;
R²⁸⁷ is —(CO)NH₂ or —OH;
R²⁹⁸ is —NH₂, —CO₂H, or —SH;
R³¹⁶ is —NH₂;
R³¹⁷ is hydrogen or —NH₂;
JJ, KK, LL, MM, NN, QQ, and RR are each independently —CH₂—;
o, p, q, r, s, t, u, v, w, x, y, z, and aa are each independently 0 or 1,
   with the proviso that o+p+q+r+s+t+u+v+w+x+y+z+aa equals 6, 7, 8, 9, 10, or 11;
cc is 0, 1, 2, 3, 4, or 5; and
bb, cc, dd, ee, ff, gg, hh, ii, jj, kk, ll, mm, nn, oo, pp, and qq are each independently 1, 2, 3, 4, or 5.

In certain embodiments of Formula II,
K is

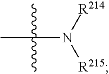

Z is

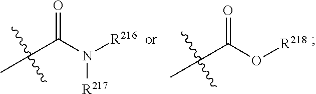

L, M, N, P, Q, R, T, U, V, W, X, and Y are each independently

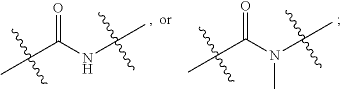

with the proviso that when
   aa is 0 and Z is not a terminal group, the terminal group is one of L, M, N, P, Q, R, T, U, V, W, X, or Y, such that one of L, M, N, P, Q, R, T, U, V, W, X, or Y, is

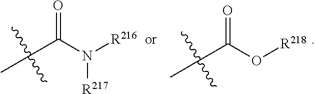

In another embodiment of Formula II,
K is

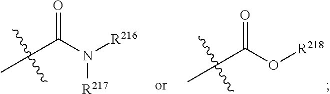

Z is

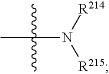

L, M, N, P, Q, R, T, U, V, W, X, and Y are each independently

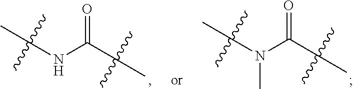

with the proviso that when
   aa is 0 and Z is not a terminal group, the terminal group is one of L, M, N, P, Q, R, T, U, V, W, X, or Y, such that one of L, M, N, P, Q, R, T, U, V, W, X, or Y, is

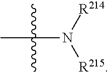

In some embodiments, the peptide of Formula II is selected from the peptides shown in Table C.

TABLE C

D-Arg-Dmt-Lys-Phe-Glu-Cys-Gly-NH₂
Phe-D-Arg-Phe-Lys-Glu-Cys-Gly-NH₂
Phe-D-Arg-Dmt-Lys-Glu-Cys-Gly-NH₂
Ala-D-Phe-D-Arg-Tyr-Lys-D-Trp-His-D-Tyr-Gly-Phe
Asp-D-Trp-Lys-Tyr-D-His-Phe-Arg-D-Gly-Lys-NH₂
D-His-Glu-Lys-Tyr-D-Phe-Arg
D-His-Lys-Tyr-D-Phe-Glu-D-Asp-D-Asp-D-His-D-
Lys-Arg-Trp-NH₂
Lys-D-Gln-Tyr-Arg-D-Phe-Trp-NH₂
Lys-Trp-D-Tyr-Arg-Asn-Phe-Tyr-D-His-NH₂
Phe-D-Arg-Lys-Trp-Tyr-D-Arg-His
Thr-Gly-Tyr-Arg-D-His-Phe-Trp-D-His-Lys
Trp-Lys-Phe-D-Asp-Arg-Tyr-D-His-Lys
Va1-D-Lys-His-Tyr-D-Phe-Ser-Tyr-Arg-NH₂
Gly-D-Phe-Lys-Tyr-His-D-Arg-Tyr-NH₂
Asp-D-Trp-Lys-Tyr-D-His-Phe-Arg-D-Gly-Lys-NH₂
D-His-Lys-Tyr-D-Phe-Glu-D-Asp-D-His-D-Lys-Arg-Trp-NH₂
H-Phe-D-Arg-Phe-Lys-Glu-Cys-Gly-NH₂
Phe-Arg-Phe-Lys-Glu-Cys-Gly
H-D-Arg-Dmt-Lys-Phe-Sar-Gly-Cys-NH₂

In another embodiment the peptide is defined by Formula III:

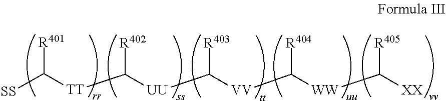

wherein:
one of SS and XX is

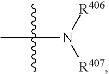

and the other is

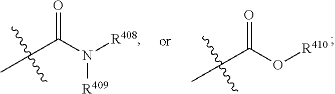

TT, UU, VV, and WW are each

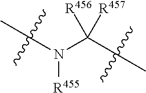

or TT, UU, VV, and WW are each

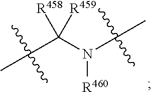

with the proviso when vv is 0 and uu is 1, one of SS and WW is

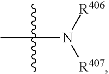

and the other of SS and WW is

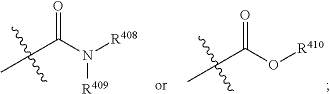

R⁴⁰¹ is

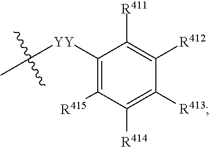

R⁴⁰² is

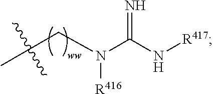

R⁴⁰³ is

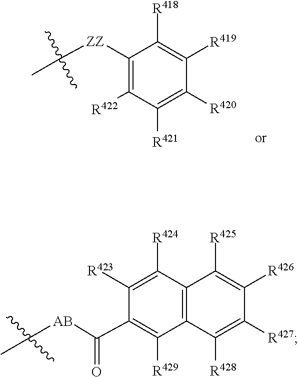

R⁴⁰⁴ is

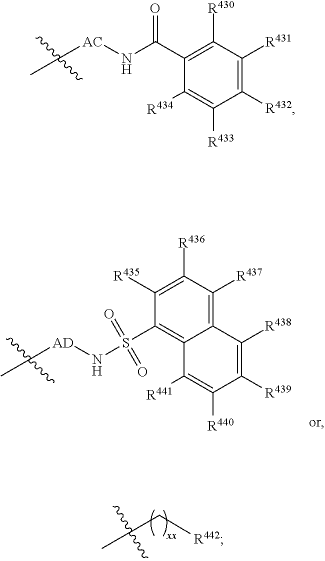

R⁴⁰⁵ is

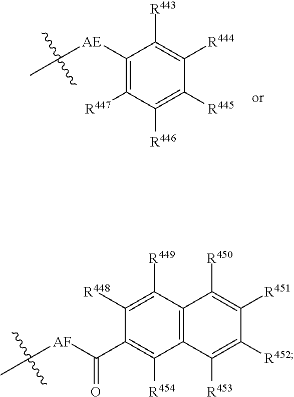

wherein
   R⁴⁰⁶, R⁴⁰⁷, R⁴⁰⁸, R⁴⁰⁹, and R⁴¹⁰ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heterobicycyl, heteroaryl, or amino protecting group; or R⁴⁰⁶ and R⁴⁰⁷ together form a 3-, 4-, 5-, 6-, 7-, or 8-member substituted or unsubstituted heterocycyl ring;
   R⁴⁵⁵ and R⁴⁶⁰ are at each occurrence independently a hydrogen, —C(O)R^{e}, or an unsubstituted C₁-C₆ alkyl group;
   R⁴⁵⁶ and R⁴⁵⁷ are each independently a hydrogen or substituted or unsubstituted C₁-C₆ alkyl group; or together R⁴⁵⁶ and R⁴⁵⁷ are C═O;
   R⁴⁵⁸ and R⁴⁵⁹ are each independently a hydrogen or substituted or unsubstituted C₁-C₆ alkyl group; or together R⁴⁵⁸ and R⁴⁵⁹ are C═O;
   R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵¹, R⁴⁵², R⁴⁵³, and R⁴⁵⁴ are each independently a hydrogen, deuterium, amino, amido, —NO₂, —CN, —OR^{e}, —SR^{e}, —NR^{e}R^{e}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{e}, C₁-C₄ alkylamino, C₁-C₄ dialkylamino, or perhaloalkyl group;
   R⁴¹⁶ and R⁴¹⁷ are each independently a hydrogen, —C(O)R^{e}, or a substituted or unsubstituted C₁-C₆ alkyl;
   R⁴⁴² is a hydrogen, —OR^{e}, —SR^{e}, —NR^{e}R^{e}, —NR^{e}R^{f}, —CO₂R^{e}, —C(O)NR^{e}R^{e}, —NR^{e}C(O)R^{e}, —NR^{e}C(NH)NH₂, —NR^{e}-dansyl, or a substituted or unsubstituted alkyl, aryl, or aralkyl group;
   YY, ZZ, and AE are each independently absent, —NH(CO)—, or —CH₂—;
   AB, AC, AD, and AF are each independently absent or C₁-C₆ alkylene group;
   R^{e} at each occurrence is independently a hydrogen or a substituted or unsubstituted C₁-C₆ alkyl group;
   R^{f} at each occurrence is independently a C₁-C₆ alkylene-NR^{e}-dansyl or C₁-C₆ alkylene-NR^{e}-anthraniloyl group;
   rr, ss, and vv are each independently 0 or 1; tt and uu are each 1 with the proviso that rr+ss+tt+uu+vv equals 4 or 5; and
   ww and xx are each independently 1, 2, 3, 4, or 5.

In some embodiments of peptides of Formula III,
R⁴⁰⁶ is a hydrogen, substituted or unsubstituted C₁-C₆ alkyl group,

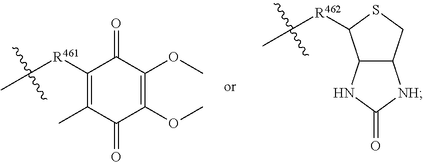

wherein R⁴⁶¹ is a —C₁-C₁₀ alkylene-CO₂— or —CO₂—C₁-C₁₀ alkylene-CO₂—; and
   R⁴⁶² is C₁-C₁₀ alkylene or C₁-C₁₀ alkylene-CO₂—;
R⁴⁰⁷, R⁴⁰⁸, R⁴⁰⁹, and R⁴¹⁰ are each independently a hydrogen or substituted or unsubstituted C₁-C₆ alkyl group;
R⁴⁵⁵ and R⁴⁶⁰ are each independently a hydrogen, —C(O)—C₁-C₆ alkyl, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)R^{e};
R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴⁴³, R⁴⁴⁴, R⁴⁴⁵, R⁴⁴⁶, and R⁴⁴⁷, are each independently a hydrogen, deuterium, methyl, or —OR^{e} group;
R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴⁸, R⁴⁴⁹, R⁴⁵⁰, R⁴⁵¹, R⁴⁵², R⁴⁵³, and R⁴⁵⁴ are each independently a hydrogen, NR^{e}R^{e}, or substituted or unsubstituted C₁-C₆ alkyl group;
R⁴⁴² is a —NR^{e}R^{e};
YY, ZZ, and AE are each independently absent or —CH₂—,
AB, AC, AD, and AF are each independently absent or C₁-C₄ alkylene group;
R^{e} at each occurrence is independently a hydrogen or a substituted or unsubstituted C₁-C₆ alkyl group;
rr, ss, and vv are each independently 0 or 1; tt and uu are each 1 with the proviso that rr+ss+tt+uu+vv equals 4 or 5; and
ww and xx are each independently 1, 2, 3, 4, or 5.

In some embodiments of peptides of Formula III,
R⁴⁰⁶ is

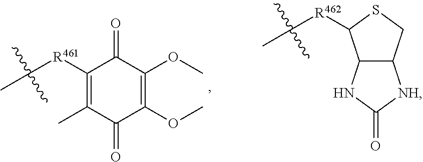

hydrogen, or methyl, wherein R⁴⁶¹ is a —(CH₂)₃—CO₂—, —(CH₂)₉—CO₂—, or —CO₂—(CH₂)₂—CO₂— and R⁴⁶² is —(CH₂)₄—CO₂—;
R⁴⁰⁷, R⁴⁰⁸, R⁴⁰⁹, and R⁴¹⁰ are each a hydrogen or methyl group;
R⁴⁵⁵ and R⁴⁶⁰ are each independently a hydrogen, —C(O)CH₃, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)CH₃;
R⁴²⁶, R⁴³⁸, and R⁴⁵⁰ are each —N(CH₃)₂;
R⁴³⁴ and R⁴⁴² are each —NH₂;
R⁴²³, R⁴²⁴, R⁴²⁵, R⁴²⁷, R⁴²⁸, R⁴²⁹, R⁴³⁰, R⁴³¹, R⁴³², R⁴³³, R⁴³⁵, R⁴³⁶, R⁴³⁷, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵², R⁴⁵³, and R⁴⁵⁴ are each hydrogen;
R⁴¹², R⁴¹⁴, R⁴¹⁹, and R⁴²¹ are each independently hydrogen or deuterium;
R⁴¹¹, R⁴¹⁵, R⁴¹⁸, and R⁴²² are each independently hydrogen, deuterium, or methyl;
R⁴¹³ and R⁴²⁰ are each independently hydrogen, deuterium, or OR^{e};
YY, ZZ, and AE are each independently —CH₂—;
AB, AC, AD, and AF are each —CH₂— or a butylene group;
R^{e} at each occurrence is independently a hydrogen or a substituted or unsubstituted C₁-C₆ alkyl group;
rr, ss, and vv are each independently 0 or 1; tt and uu are each 1
   with the proviso that rr+ss+tt+uu+vv equals 4 or 5; and
ww and xx are each independently 3 or 4.

In certain embodiments of Formula III,

SS is

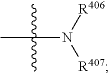

XX is

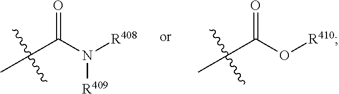

TT, UU, VV, and WW are each independently

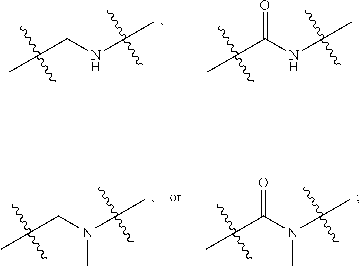

with the proviso when vv is 0 and uu is 1, WW is

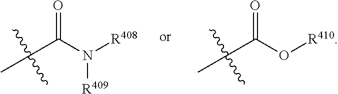

In some embodiments, the peptide of Formula III is selected from the peptides shown in Table D.

TABLE D

6-Butyric acid CoQ0-Phe-D-Arg-Phe-Lys-NH₂
6-Decanoic acid CoQ0-Phe-D-Arg-Phe-Lys-NH₂
H-D-N2-acetylarginine-Dmt-Lys-Phe-NH₂
H-D-N8-acetylarginine-Dmt-Lys-Phe-NH₂
H-N2-acetyl-D-arginine-L-Dmt-L-Lys-L-Phe-NH₂
H-N7-acetyl-D-arginine-Dmt-Lys-Phe-NH₂
H-Phe(d5)-D-Arg-Phe(d5)-Lys-NH₂
Succinic monoester CoQ0-Phe-D-Arg-Phe-Lys-HN₂
Dmt-D-Arg-Phe-(atn)Dap-NH₂
Dmt-D-Arg-Phe-(dns)Dap-NH₂
Dmt-D-Arg-Ald-Lys-NH₂
Dmt-D-Arg-Phe-Lys-Ald-NH₂
Bio-2′6′Dmt-D-Arg-Phe-Lys-NH₂
2′6′Dmt-D-Arg-Phe-dnsDap-NH₂
2′6′Dmt-D-Arg-Phe-atnDap-NH₂
H-D-Arg-Ψ[CH₂-NH]Dmt-Lys-Phe-NH₂
H-D-Arg-Dmt-Ψ[CH₂-NH]Lys-Phe-NH₂
H-D-Arg-Dmt-LysΨ[CH₂-NH]Phe-NH₂
H-D-Arg-Dmt-Ψ[CH₂-NH]Lys-Ψ[CH₂-NH]Phe-NH₂

In some embodiments, the peptide is selected from the peptides shown in Table E.

TABLE E

Arg-D-Leu-D-Tyr-Phe-Lys-Glu-D-Lys-Arg-D-Trp-Lys-D-Phe-
Tyr-D-Arg-Gly
Asp-Arg-D-Phe-Cys-Phe-D-Arg-D-Lys-Tyr-Arg-D-Tyr-Trp-D-
His-Tyr-D-Phe-Lys-Phe
D-Glu-Asp-Lys-D-Arg-D-His-Phe-Phe-D-Val-Tyr-Arg-Tyr-D-
Tyr-Arg-His-Phe-NH₂
Glu-Arg-D-Lys-Tyr-D-Val-Phe-D-His-Trp-Arg-D-Gly-Tyr-Arg-
D-Met-NH₂
Gly-Ala-Lys-Phe-D-Lys-Glu-Arg-Tyr-His-D-Arg-D-Arg-Asp-
Tyr-Trp-D-His-Trp-His-D-Lys-Asp
His-Tyr-D-Arg-Trp-Lys-Phe-D-Asp-Ala-Arg-Cys-D-Tyr-His-
Phe-D-Lys-Tyr-His-Ser-NH₂
Phe-Phe-D-Tyr-Arg-Glu-Asp-D-Lys-Arg-D-Arg-His-Phe-NH₂
Phe-Tyr-Lys-D-Arg-Trp-His-D-Lys-D-Lys-Glu-Arg-D-Tyr-Thr
Thr-Tyr-Arg-D-Lys-Trp-Tyr-Glu-Asp-D-Lys-D-Arg-His-Phe-D-
Tyr-Gly-Val-Ile-D-His-Arg-Tyr-Lys-NH₂
Tyr-Asp-D-Lys-Tyr-Phe-D-Lys-D-Arg-Phe-Pro-D-Tyr-His-Lys
Tyr-D-His-Phe-D-Arg-Asp-Lys-D-Arg-His-Trp-D-His-Phe
Phe-Tyr-Lys-D-Arg-Trp-His-D-Lys-D-Lys-Glu-Arg-D-Tyr-Thr
Tyr-Asp-D-Lys-Tyr-Phe-D-Lys-D-Arg-Phe-Pro-D-Tyr-His-Lys
Glu-Arg-D-Lys-Tyr-D-Val-Phe-D-His-Trp-Arg-D-Gly-Tyr-Arg-
D-Met-NH₂
Arg-D-Leu-D-Tyr-Phe-Lys-Glu-D-Lys-Arg-D-Trp-Lys-D-Phe-
Tyr-D-Arg-Gly
Gly-Ala-Lys-Phe-D-Lys-Glu-Arg-Tyr-His-D-Arg-D-Arg-Asp-
Tyr-Trp-D-His-Trp-His-D-Lys-Asp
Gly-Ala-Lys-Phe-D-Lys-Glu-Arg-Tyr-His-D-Arg-D-Arg-Asp-
Tyr-Trp-D-His-Trp-His-D-Lys-Asp

In one embodiment, the aromatic-cationic peptides of the present technology have a core structural motif of alternating aromatic and cationic amino acids. For example, the peptide may be a tetrapeptide defined by any of Formulas A to F set forth below:
Aromatic-Cationic-Aromatic-Cationic (Formula A)
Cationic-Aromatic-Cationic-Aromatic (Formula B)
Aromatic-Aromatic-Cationic-Cationic (Formula C)
Cationic-Cationic-Aromatic-Aromatic (Formula D)
Aromatic-Cationic-Cationic-Aromatic (Formula E)
Cationic-Aromatic-Aromatic-Cationic (Formula F)

wherein, Aromatic is a residue selected from the group consisting of: Phe (F), Tyr (Y), and Trp (W). In some embodiments, the Aromatic residue may be substituted with a saturated analog of an aromatic residue, e.g., Cyclohexylalanine (Cha). In some embodiments, Cationic is a residue selected from the group consisting of: Arg (R), Lys (K), and His (H).

The amino acids of the aromatic-cationic peptides of the present technology can be any amino acid. As used herein, the term “amino acid” is used to refer to any organic molecule that contains at least one amino group and at least one carboxyl group. In some embodiments, at least one amino group is at the α position relative to the carboxyl group.

The amino acids may be naturally occurring. Naturally occurring amino acids include, for example, the twenty most common levorotatory (L) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val).

Other naturally occurring amino acids include, for example, amino acids that are synthesized in metabolic processes not associated with protein synthesis. For example, the amino acids ornithine and citrulline are synthesized in mammalian metabolism during the production of urea.

The peptides useful in the present technology can contain one or more non-naturally occurring amino acids. The non-naturally occurring amino acids may be (L-), dextrorotatory (D-), or mixtures thereof. In some embodiments, the peptide has no amino acids that are naturally occurring.

Non-naturally occurring amino acids are those amino acids that typically are not synthesized in normal metabolic processes in living organisms, and do not naturally occur in proteins. In certain embodiments, the non-naturally occurring amino acids useful in the present technology are also not recognized by common proteases.

The non-naturally occurring amino acid can be present at any position in the peptide. For example, the non-naturally occurring amino acid can be at the N terminus, the C-terminus, or at any position between the N-terminus and the C-terminus.

The non-natural amino acids may, for example, comprise alkyl, aryl, or alkylaryl groups. Some examples of alkyl amino acids include α-aminobutyric acid, β-aminobutyric acid, γ-aminobutyric acid, δ-aminovaleric acid, and ε-aminocaproic acid. Some examples of aryl amino acids include ortho-, meta, and para-aminobenzoic acid. Some examples of alkylaryl amino acids include ortho-, meta-, and para-aminophenyl acetic acid, and γ-phenyl-β-aminobutyric acid.

Non-naturally occurring amino acids also include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva), norleucine (Nle), and hydroxyproline (Hyp).

Another example of a modification of an amino acid in a peptide useful in the present methods is the derivatization of a carboxyl group of an aspartic acid or a glutamic acid residue of the peptide. One example of derivatization is amidation with ammonia or with a primary or secondary amine, e.g., methylamine, ethylamine, dimethylamine or diethylamine. Another example of derivatization includes esterification with, for example, methyl or ethyl alcohol.

Another such modification includes derivatization of an amino group of a lysine, arginine, or histidine residue. For example, such amino groups can be alkylated or acylated. Some suitable acyl groups include, for example, a benzoyl group or an alkanoyl group comprising any of the C₁-C₄ alkyl groups mentioned above, such as an acetyl or propionyl group.

In some embodiments, the non-naturally occurring amino acids are resistant, and in some embodiments insensitive, to common proteases. Examples of non-naturally occurring amino acids that are resistant or insensitive to proteases include the dextrorotatory (D-) form of any of the above-mentioned naturally occurring L-amino acids, as well as L- and/or D non-naturally occurring amino acids. The D-amino acids do not normally occur in proteins, although they are found in certain peptide antibiotics that are synthesized by means other than the normal ribosomal protein synthetic machinery of the cell, as used herein, the D-amino acids are considered to be non-naturally occurring amino acids.

In order to minimize protease sensitivity, the peptides useful in the methods of the present technology should have less than five, less than four, less than three, or less than two contiguous L-amino acids recognized by common proteases, irrespective of whether the amino acids are naturally or non-naturally occurring. In some embodiments, the peptide has only D-amino acids, and no L-amino acids.

If the peptide contains protease sensitive sequences of amino acids, at least one of the amino acids is a non-naturally-occurring D-amino acid, thereby conferring protease resistance. An example of a protease sensitive sequence includes two or more contiguous basic amino acids that are readily cleaved by common proteases, such as endopeptidases and trypsin. Examples of basic amino acids include arginine, lysine and histidine. In some embodiments, at least one of the amides in the peptide backbone are alkylated, thereby conferring protease resistance.

It is important that the aromatic-cationic peptides have a minimum number of net positive charges at physiological pH in comparison to the total number of amino acid residues in the peptide. The minimum number of net positive charges at physiological pH is referred to below as (pₘ). The total number of amino acid residues in the peptide is referred to below as (r).

The minimum number of net positive charges discussed below are all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

Typically, a peptide has a positively charged N-terminal amino group and a negatively charged C-terminal carboxyl group. The charges cancel each other out at physiological pH. As an example of calculating net charge, the peptide Tyr-Arg-Phe-Lys-Glu-His-Trp-Arg has one negatively charged amino acid (i.e., Glu) and four positively charged amino acids (i.e., two Arg residues, one Lys, and one His). Therefore, the above peptide has a net positive charge of three.

In one embodiment, the aromatic-cationic peptides have a relationship between the minimum number of net positive charges at physiological pH (pₘ) and the total number of amino acid residues (r) wherein 3pₘ is the largest number that is less than or equal to r+1. In this embodiment, the relationship between the minimum number of net positive charges (pₘ) and the total number of amino acid residues (r) is as follows:

TABLE 1
Amino acid number and net positive charges (3pₘ ≤ p + 1)

| | (r) | | | | | | | | | | | | | | | | | |
|---|---|---|---|---|---|---|---|---|---|---|---|---|---|---|---|---|---|---|
| | 3 | 4 | 5 | 6 | 7 | 8 | 9 | 10 | 11 | 12 | 13 | 14 | 15 | 16 | 17 | 18 | 19 | 20 |
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of net positive charges (pₘ) and the total number of amino acid residues (r) wherein 2pₘ is the largest number that is less than or equal to r+1. In this embodiment, the relationship between the minimum number of net positive charges (pₘ) and the total number of amino acid residues (r) is as follows:

TABLE 2
Amino acid number and net positive charges (2pₘ ≤ p + 1)

| | (r) | | | | | | | | | | | | | | | | | |
|---|---|---|---|---|---|---|---|---|---|---|---|---|---|---|---|---|---|---|
| | 3 | 4 | 5 | 6 | 7 | 8 | 9 | 10 | 11 | 12 | 13 | 14 | 15 | 16 | 17 | 18 | 19 | 20 |
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of amino acid residues (r) are equal. In another embodiment, the peptides have three or four amino acid residues and a minimum of one net positive charge, or a minimum of two net positive charges, or a minimum of three net positive charges.

It is also important that the aromatic-cationic peptides have a minimum number of aromatic groups in comparison to the total number of net positive charges (pₜ). The minimum number of aromatic groups will be referred to below as (a). Naturally-occurring amino acids that have an aromatic group include the amino acids histidine, tryptophan, tyrosine, and phenylalanine. For example, the hexapeptide Lys-Gln-Tyr-D-Arg-Phe-Trp has a net positive charge of two (contributed by the lysine and arginine residues) and three aromatic groups (contributed by tyrosine, phenylalanine and tryptophan residues).

The aromatic-cationic peptides should also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≤ pₜ + 1 or a = pₜ = 1)

| | (Pₜ) | | | | | | | | | | | | | | | | | | | |
|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of aromatic groups (a) and the total number of net positive charges (pₜ) wherein 2a is the largest number that is less than or equal to pₜ+1. In this embodiment, the relationship between the minimum number of aromatic amino acid residues (a) and the total number of net positive charges (pₜ) is as follows:

TABLE 4
Aromatic groups and net positive charges (2a ≤ pₜ + 1 or a = pₜ = 1)

| | (pₜ ) | | | | | | | | | | | | | | | | | | | |
|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pt) are equal.

In some embodiments, carboxyl groups, especially the terminal carboxyl group of a C-terminal amino acid, are amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine. Accordingly, the amino acid at the C-terminus of the peptide may be converted to an amido, N-methylamido, N-ethylamido, N,N-dimethylamido, N,N-diethyl amido, N-methyl-N-ethylamido, N-phenylamido or N-phenyl-N-ethylamido group.

The free carboxylate groups of the asparagine, glutamine, aspartic acid, and glutamic acid residues not occurring at the C-terminus of the aromatic-cationic peptides of the present technology may also be amidated wherever they occur within the peptide. The amidation at these internal positions may be with ammonia or any of the primary or secondary amines described herein.

In one embodiment, the aromatic-cationic peptide useful in the methods of the present technology is a tripeptide having two net positive charges and at least one aromatic amino acid. In a particular embodiment, the aromatic-cationic peptide useful in the methods of the present technology is a tripeptide having two net positive charges and two aromatic amino acids.

In some embodiments, the aromatic-cationic peptide is a peptide having:
at least one net positive charge;
a minimum of four amino acids;
a maximum of about twenty amino acids;
a relationship between the minimum number of net positive charges (pₘ) and the total number of amino acid residues (r) wherein 3pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1.

In one embodiment, 2pₘ is the largest number that is less than or equal to r+1, and a may be equal to pₜ. The aromatic-cationic peptide may be a water-soluble peptide having a minimum of two or a minimum of three positive charges.

In one embodiment, the peptide comprises one or more non-naturally occurring amino acids, for example, one or more D-amino acids. In some embodiments, the C-terminal carboxyl group of the amino acid at the C-terminus is amidated. In certain embodiments, the peptide has a minimum of four amino acids. The peptide may have a total of about 6, a total of about 9, or a total of about 12 amino acids.

In one embodiment, the peptides have a tyrosine residue or a tyrosine derivative at the N-terminus (i.e., the first amino acid position). Suitable derivatives of tyrosine include 2′-methyltyrosine (Mmt); 2′,6′-dimethyltyrosine (2′6′-Dmt); 3′,5′-dimethyltyrosine (3′5′Dmt); N,2′,6′-trimethyltyrosine (Tmt); and 2′-hydroxy-6′-methyltyrosine (Hmt).

In one embodiment, a peptide has the formula Tyr-D-Arg-Phe-Lys-NH₂. Tyr-D-Arg-Phe-Lys-NH₂ has a net positive charge of three, contributed by the amino acids tyrosine, arginine, and lysine and has two aromatic groups contributed by the amino acids phenylalanine and tyrosine. The tyrosine of Tyr-D-Arg-Phe-Lys-NH₂ can be a modified derivative of tyrosine such as in 2′,6′-dimethyltyrosine to produce the compound having the formula 2′,6′-Dmt-D-Arg-Phe-Lys-NH₂. 2′,6′-Dmt-D-Arg-Phe-Lys-NH₂ has a molecular weight of 640 and carries a net three positive charge at physiological pH. 2′,6′-Dmt-D-Arg-Phe-Lys-NH₂ readily penetrates the plasma membrane of several mammalian cell types in an energy-independent manner (Zhao et al., J. Pharmacol Exp Ther., 304:425-432, 2003).

Alternatively, in some embodiments, the aromatic-cationic peptide does not have a tyrosine residue or a derivative of tyrosine at the N-terminus (i.e., amino acid position 1). The amino acid at the N-terminus can be any naturally-occurring or non-naturally-occurring amino acid other than tyrosine. In one embodiment, the amino acid at the N-terminus is phenylalanine or its derivative. Exemplary derivatives of phenylalanine include 2′-methylphenylalanine (Mmp), 2′,6′-dimethylphenylalanine (2′,6′-Dmp), N,2′,6′-trimethylphenylalanine (Tmp), and 2′-hydroxy-6′-methylphenylalanine (Hmp).

An example of an aromatic-cationic peptide that does not have a tyrosine residue or a derivative of tyrosine at the N-terminus is a peptide with the formula Phe-D-Arg-Phe-Lys-NH₂. Alternatively, the N-terminal phenylalanine can be a derivative of phenylalanine such as 2′,6′-dimethylphenylalanine (2′6′-Dmp). In one embodiment, the amino acid sequence of 2′,6′-Dmt-D-Arg-Phe-Lys-NH₂ is rearranged such that Dmt is not at the N-terminus. An example of such an aromatic-cationic peptide is a peptide having the formula of D-Arg-2′6′-Dmt-Lys-Phe-NH₂.

Suitable substitution variants of the peptides listed herein include conservative amino acid substitutions. Amino acids may be grouped according to their physicochemical characteristics as follows:
(a) Non-polar amino acids: Ala(A) Ser(S) Thr(T) Pro(P) Gly(G) Cys (C);
(b) Acidic amino acids: Asn(N) Asp(D) Glu(E) Gln(Q);
(c) Basic amino acids: His(H) Arg(R) Lys(K);
(d) Hydrophobic amino acids: Met(M) Leu(L) Ile(I) Val(V); and
(e) Aromatic amino acids: Phe(F) Tyr(Y) Trp(W).

Substitutions of an amino acid in a peptide by another amino acid in the same group are referred to as a conservative substitution and may preserve the physicochemical characteristics of the original peptide. In contrast, substitutions of an amino acid in a peptide by another amino acid in a different group are generally more likely to alter the characteristics of the original peptide.

The amino acids of the peptides disclosed herein may be in either the L- or the D-configuration.

### III. USES OF PEPTIDE CONJUGATES TO TREAT OR PREVENT COMPLEX REGIONAL PAIN SYNDROME

In some aspects, the present technology provides methods for treating, ameliorating, or preventing complex regional pain syndrome in a subject diagnosed as having, suspected as having, or at risk of having complex regional pain syndrome.

Complex regional pain syndrome (CRPS) is a chronic pain condition most often affecting one of the limbs (arms, legs, hands, or feet), usually after a disease, injury, or trauma to that limb. CRPS may develop as a consequence of a lesion, damage, or disease affecting the somatosensory pathways in the peripheral or central nervous system. CRPS is divided into two types Type I (CRPS-I) and Type II (CRPS-II). CRPS-I does not exhibit demonstrable nerve lesion and can occur after soft-tissue or bone injury. CRPS-II exhibits obvious nerve damage or injury.

In some aspects, the present technology provides methods for treating complex regional pain syndrome (e.g., CRPS-I) in a subject in need thereof. In some embodiments, the method comprises administering a therapeutically effective amount of one or more peptide conjugates, wherein the peptide conjugates comprise an aromatic-cationic peptide, such as, e.g., 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂, or pharmaceutically acceptable salt thereof, such as acetate, tartrate, or trifluoroacetate salt, conjugated to an antioxidant selected from TEMPO, Trolox, PBN, AHDP, DBHP, Caf, and Hcm, to the subject thereby treating CRPS. In some therapeutic applications, one or more peptide conjugates are administered to a subject suspected of, or already suffering from CRPS in an amount sufficient to cure, or at least partially arrest or ameliorate, the symptoms of the disease, including its complications and intermediate pathological phenotypes in development of the disease.

Administering peptide conjugates of aromatic peptides and the disclosed antioxidants (e.g., TEMPO, Trolox, PBN, AHDP, DBHP, Caf, and Hcm) results in a synergistic biological effect when administered in a therapeutically effective amount to a subject suffering from CRPS and in need of treatment. An advantage of the peptide conjugate is that lower doses of aromatic-cationic peptide and/or disclosed antioxidants may be needed to prevent, ameliorate or treat CRPS in a subject. Further, potential side-effects of treatment may be avoided by use of lower dosages of aromatic-cationic peptide and/or the disclosed antioxidant. In some embodiments, the therapy comprises administering to a subject in need thereof at least one peptide conjugate disclosed herein.

Subjects suffering from CRPS can be identified by any or a combination of diagnostic or prognostic assays known in the art. By way of example, but not by way of limitation, symptoms of CRPS include, but are not limited to, shooting and/or burning pain, tingling and/or numbness, neurogenic inflammation, nociceptive sensitisation, vasomotor dysfunction, and maladaptive neuroplasticity in or near afflicted region, allodynia, hyperalgesia, systemic autonomic dysregulation, neurogenic edema, and changes in urological or gastrointestinal function.

In prophylactic applications, the peptide conjugates of the present technology are administered to a subject susceptible to, or otherwise at risk of CRSP in an amount sufficient to eliminate or reduce the risk, or delay the onset of CRSP, including biochemical, histological and/or behavioral symptoms of the disease, its complications and intermediate pathological phenotypes presenting during development of the disease. Administration of a prophylactic peptide conjugates of the present technology can occur prior to the manifestation of symptoms characteristic of the aberrancy, such that CRPS is prevented or, alternatively, delayed in its progression. By way of example, but not by way of limitation, in some embodiments, administration of one or more peptide conjugates described herein, eliminates or reduces the risk, or delays the onset one or more symptoms of CRPS, including, but not limited to, shooting and/or burning pain, tingling and/or numbness, neurogenic inflammation, nociceptive sensitisation, vasomotor dysfunction, and maladaptive neuroplasticity in or near afflicted region, allodynia, hyperalgesia, systemic autonomic dysregulation, neurogenic edema, and changes in urological or gastrointestinal function.

In some embodiments, an effective dose of the peptide conjugates described herein (e.g., an aromatic-cationic peptide such as 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂, or pharmaceutically acceptable salt thereof, conjugated to an antioxidant selected from TEMPO, Trolox, PBN, AHDP, DBHP, Caf, and Hcm), can be administered via a variety of routes including, but not limited to, e.g., parenteral via an intravenous infusion given as repeated bolus infusions or constant infusion, intradermal injection, subcutaneously given as repeated bolus injection or constant infusion, or oral administration.

In certain embodiments, an effective parenteral dose (given intravenously, intraperitoneally, or subcutaneously) of peptide conjugates of the present technology to an experimental animal is within the range of 2 mg/kg up to 160 mg/kg body weight, or 10 mg/kg, or 30 mg/kg, or 60 mg/kg, or 90 mg/kg, or 120 mg/kg body weight.

In some embodiments, an effective parenteral dose (given intravenously, intraperitoneally, or subcutaneously) of peptide conjugates of the present technology to an experimental animal can be administered three times weekly, twice weekly, once weekly, once every two weeks, once monthly, or as a constant infusion.

In certain embodiments, an effective parental dose (given intravenously or subcutaneously) of peptide conjugates of the present technology to a human subject is within the range of 0.5 mg/kg up to 25 mg/kg body weight, or 1 mg/kg, or 2 mg/kg, or 5 mg/kg or 7.5 mg/kg, or 10 mg/kg body weight, or 15 mg/kg body weight.

In some embodiments, an effective parental dose (given intravenously or subcutaneously) of peptide conjugates of the present technology to a human subject can be administered three times weekly, twice weekly, once weekly, once every two weeks, once monthly, or as a constant infusion.

Any method known to those in the art for contacting a cell, organ or tissue with a peptide conjugate of the present technology may be employed. Suitable methods include in vitro, ex vivo, or in vivo methods. In vivo methods typically include the administration of peptide conjugates of the present technology, such as those described herein, to a mammal, such as a human. When used in vivo for therapy, a peptide conjugate of the present technology is administered to the subject in effective amounts (i.e., amounts that have desired therapeutic effect). Compositions will normally be administered parenteral, topically, or orally. The dose and dosage regimen will depend upon the type and severity of disease or injury, the characteristics of the particular peptide conjugate of the present technology e.g., its therapeutic index, the characteristics of the subject, and the subject's medical history.

In some embodiments, the dosage of the peptide conjugate of the present technology is provided at a “low,” “mid,” or “high” dose level. In some embodiments, the low dose is from about 0.001 to about 0.5 mg/kg/h, or from about 0.01 to about 0.1 mg/kg/h. In some embodiments, the mid-dose is from about 0.1 to about 1.0 mg/kg/h, or from about 0.1 to about 0.5 mg/kg/h. In some embodiments, the high dose is from about 0.5 to about 10 mg/kg/h, or from about 0.5 to about 2 mg/kg/h. The skilled artisan will appreciate that certain factors may influence the dosage and timing required to effectively treat a subject, including but not limited to, the severity of the medical disease or condition, previous treatments, the general health and/or age of the subject, and other diseases present. Moreover, treatment of a subject with a therapeutically effective amount of the peptide conjugates described herein can include a single treatment or a series of treatments.

### Determination of the Biological Effect of Peptide Conjugates of the Present Technology

In various embodiments, suitable in vitro or in vivo assays are performed to determine the effect of a specific composition of the present technology and whether its administration is indicated for treatment. In various embodiments, in vitro assays can be performed with representative animal models, to determine if a peptide conjugate-based therapeutic exerts the desired effect in treating a disease or condition. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

### IV. SYNTHESIS OF COMPOSITIONS OF THE PRESENT TECHNOLOGY

The compounds useful in the methods of the present disclosure (e.g., peptide conjugate of the present technology) may be synthesized by any method known in the art.

The aromatic-cationic peptides disclosed herein (such as 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂) may be synthesized by any method known in the art. Exemplary, non-limiting methods for chemically synthesizing the protein include those described by Stuart and Young in “Solid Phase Peptide Synthesis,” Second Edition, Pierce Chemical Company (1984), and in “Solid Phase Peptide Synthesis,” Methods Enzymol. 289, Academic Press, Inc, New York (1997).

Recombinant peptides may be generated using conventional techniques in molecular biology, protein biochemistry, cell biology, and microbiology, such as those described in Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

Aromatic-cationic peptide precursors may be made by either chemical (e.g., using solution and solid phase chemical peptide synthesis) or recombinant syntheses known in the art. Precursors of e.g., amidated aromatic-cationic peptides of the present technology may be made in like manner. In some embodiments, recombinant production is believed significantly more cost effective. In some embodiments, precursors are converted to active peptides by amidation reactions that are also known in the art. For example, enzymatic amidation is described in U.S. Pat. No. 4,708,934 and European Patent Publications 0 308 067 and 0 382 403. Recombinant production can be used for both the precursor and the enzyme that catalyzes the conversion of the precursor to the desired active form of the aromatic-cationic peptide. Such recombinant production is discussed in Biotechnology, Vol. 11 (1993) pp. 64-70, which further describes a conversion of a precursor to an amidated product. During amidation, a keto-acid such as an alpha-keto acid, or salt or ester thereof, wherein the alpha-keto acid has the molecular structure RC(O)C(O)OH, and wherein R is selected from the group consisting of aryl, a C₁-C₄ hydrocarbon moiety, a halogenated or hydroxylated C₁-C₄ hydrocarbon moiety, and a C₁-C₄ carboxylic acid, may be used in place of a catalase co-factor. Examples of these keto acids include, but are not limited to, ethyl pyruvate, pyruvic acid and salts thereof, methyl pyruvate, benzoyl formic acid and salts thereof, 2-ketobutyric acid and salts thereof, 3-methyl-2-oxobutanoic acid and salts thereof, and 2-keto glutaric acid and salts thereof.

In some embodiments, the production of the recombinant aromatic-cationic peptide may proceed, for example, by producing glycine-extended precursor in E. coli as a soluble fusion protein with glutathione-S-transferase. An α-amidating enzyme catalyzes conversion of precursors to active aromatic-cationic peptide. That enzyme is recombinantly produced, for example, in Chinese Hamster Ovary (CHO) cells as described in the Biotechnology article cited above. Other precursors to other amidated peptides may be produced in like manner. Peptides that do not require amidation or other additional functionalities may also be produced in like manner. Other peptide active agents are commercially available or may be produced by techniques known in the art.

### V. PREPARATION OF THE PEPTIDE CONJUGATES OF THE PRESENT TECHNOLOGY

In some embodiments, an antioxidant selected from TEMPO, Trolox, PBN, AHDP, DBHP, Caf, and Hcm and an aromatic-cationic peptide as described herein (e.g., 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂, or pharmaceutically acceptable salt thereof) associate to form a peptide conjugate of the present technology. The antioxidant and aromatic-cationic peptide can associate by any method known to those in the art. Suitable types of associations involve covalent bond formation.

For covalent bond formation, a functional group on the antioxidant typically associates with a functional group on the aromatic-cationic peptide. Alternatively, a functional group on the aromatic-cationic peptide associates with a functional group on the antioxidant.

The functional groups on the antioxidant and aromatic-cationic peptide can associate directly. For example, a functional group (e.g., an aldehyde group) on an antioxidant can associate with a functional group (e.g., a primary amino group) on an aromatic-cationic peptide to form a secondary amino group by reductive amination. In another example, a functional group (e.g., a carboxylic acid group) on an antioxidant can associate with a functional group (e.g., a primary amino group) on an aromatic-cationic peptide to form an amide group.

Alternatively, the functional groups can associate through a cross-linking agent (i.e., linker). Some examples of cross-linking agents are described below. The cross-linker can be attached to either the antioxidant or the aromatic-cationic peptide.

The linker may and may not affect the number of net charges of the aromatic-cationic peptide. Typically, the linker will not contribute to the net charge of the aromatic-cationic peptide. Each amino group, if any, present in the linker will contribute to the net positive charge of the aromatic-cationic peptide. Each carboxyl group, if any, present in the linker will contribute to the net negative charge of the aromatic-cationic peptide.

The number of antioxidants or aromatic-cationic peptides in the peptide conjugate is limited by the capacity of the peptide to accommodate multiple antioxidants or the capacity of the antioxidant to accommodate multiple peptides. For example, steric hindrance may hinder the capacity of the peptide to accommodate especially large molecules. Alternatively, steric hindrance may hinder the capacity of the molecule to accommodate a relatively large (e.g., seven, eight, nine or ten amino acids in length) aromatic-cationic peptide.

The number of antioxidants or aromatic-cationic peptides in the peptide conjugate is also limited by the number of functional groups present on the other. For example, the maximum number of antioxidants associated with a peptide conjugate depends on the number of functional groups present on the aromatic-cationic peptide. Alternatively, the maximum number of aromatic-cationic peptides associated with an antioxidant depends on the number of functional groups present on the antioxidant.

In one embodiment, the peptide conjugate comprises at least one antioxidant, and in some embodiments, at least two antioxidants, associated with an aromatic-cationic peptide. A relatively large peptide (e.g., eight, ten amino acids in length) containing several (e.g., 3, 4, 5 or more) functional groups can be associated with several (e.g., 3, 4, 5 or more) antioxidants.

In another embodiment, the peptide conjugate comprises at least one aromatic-cationic peptide, and, in some embodiments, at least two aromatic-cationic peptides, associated with an antioxidant. For example, an antioxidant containing several functional groups (e.g., 3, 4, 5 or more) can be associated with several (e.g., 3, 4, or 5 or more) peptides.

In yet another embodiment, the peptide conjugate comprises one aromatic-cationic peptide associated to one antioxidant.

In one embodiment, a peptide conjugate comprises at least one antioxidant covalently bound (e.g., conjugated) to at least one aromatic-cationic peptide. The molecule can be covalently bound to an aromatic-cationic peptide by any method known to those in the art. For example, a functional group on the antioxidant may be directly attached to a functional group on the aromatic-cationic peptide. Some examples of suitable functional groups include, for example, amino, carboxyl, sulfhydryl, maleimide, isocyanate, isothiocyanate and hydroxyl. In some embodiments, a functional group on the antioxidant and a functional group on the aromatic-cationic peptide attach to form an amide. In some embodiments, a functional group on the antioxidant and a functional group on the aromatic-cationic peptide attach to form a secondary amine. In some embodiments, a functional group on the antioxidant and a functional group on the aromatic-cationic peptide attach to form a tertiary amine. In some embodiments, a functional group on the antioxidant is altered prior to interaction with a functional group on the aromatic-cationic peptide. For example, the carboxylic group of Tro or Caf is reduced to an aldehyde prior to interaction with a functional group on the aromatic-cationic peptide to attach the antioxidant to the aromatic-cationic peptide.

The antioxidant may also be covalently bound to the aromatic-cationic peptide by means of cross-linking agents, such as diamines, dialdehydes, dicarboxylic acids, carbodiimides, dimaleimides, amino carboxylic acids, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands. In some embodiments, the cross-linking agent provides a linker through which the antioxidant is indirectly conjugated to the aromatic-cationic peptide. In some embodiments, the linker may be a C₁-C₁₂ linker and may include one or more groups independently selected from the group consisting of a carbonyl, an amine, and an alkylene group. In some embodiments, the linker is selected from the group consisting of —C(O)—(C₁-C₆ alkylene)-C(O)—, —C(O)—(C₁-C₆ alkylene)-NH—, and —NH—(C₁-C₆ alkylene)-NH—. In some embodiments, the cross-linking agent enables the antioxidant to be directly conjugated to the aromatic-cationic peptide.

The functional group on the antioxidant may be different from the functional group on the peptide. For example, if a sulfhydryl group is present on the antioxidant, the antioxidant can be cross-linked to the peptide, e.g., [Dmt¹]DALDA, through the 4-amino group of lysine by using the cross-linking reagent SMCC (i.e., succinimidyl 4-(N-maleimidomethyl)cyclohexane-1-carboxylate) from Pierce Biotechnology. Accordingly, in some embodiments, the cross-linking reagent provides a linker between the peptide and the antioxidant. In another example, the 4-amino group of lysine of DALDA can be conjugated directly to a carboxylgroup on an antioxidant by using the crosslinking reagent EDC (i.e., (N-[3-dimethylaminopropyl-N′-ethylcarboiimide]) from Pierce Biotechnology.

Alternatively, the functional group on the antioxidant and peptide can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate.2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc. Accordingly, in some embodiments, the cross-linker provides a linker between the peptide and the antioxidant.

To conjugate the antioxidants and the peptides, the antioxidants, peptides, and cross-linker are typically mixed together. The order of addition of the antioxidants, peptides, and cross-linker is not important. For example, the peptide can be mixed with the cross-linker, followed by addition of the antioxidant. Alternatively, the antioxidant can be mixed with the cross-linker, followed by addition of the peptide. Optimally, the antioxidant and the peptides are mixed, followed by addition of the cross-linker.

The covalently bound peptide conjugates deliver the antioxidant and/or aromatic-cationic peptide to a cell. In some instances, the antioxidant functions in the cell without being cleaved from the aromatic-cationic peptide. For example, if the aromatic-cationic peptide does not block the catalytic site of the molecule, then cleavage of the molecule from the aromatic-cationic peptide is not necessary.

In some embodiments, the aromatic-cationic peptides and antioxidants are mixed together by any method known to those in the art. The order of mixing is not important. For instance, antioxidants can be physically mixed with modified or unmodified aromatic-cationic peptides by any method known to those in the art. Alternatively, the modified or unmodified aromatic-cationic peptides can be physically mixed with the molecules by any method known to those in the art.

In some embodiments, the aromatic-cationic peptides and antioxidants are placed in a container and agitated, by for example, shaking the container, to mix the aromatic-cationic peptides and antioxidants.

The aromatic-cationic peptides can be modified by any method known to those in the art. For instance, the aromatic-cationic peptide may be modified by means of cross-linking agents or functional groups, as described above. The linker may and may not affect the number of net charges of the aromatic-cationic peptide. Typically, the linker will not contribute to the net charge of the aromatic-cationic peptide. Each amino group, if any, present in the linker contributes to the net positive charge of the aromatic-cationic peptide. Each carboxyl group, if any, present in the linker contributes to the net negative charge of the aromatic-cationic peptide.

For example, [Dmt₁]DALDA can be modified, through the 4-amino group of lysine by using the cross-linking reagent SMCC (i.e., succinimidyl 4-(N-maleimidomethyl)cyclohexane-1-carboxylate) from Pierce Biotechnology. To form a peptide conjugate, the modified aromatic-cationic peptide is usually formed first and then mixed with the antioxidant.

In some embodiments, at least one antioxidant and at least one aromatic-cationic peptide as described above (e.g., 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂ or a pharmaceutically acceptable salt thereof), are associated to form a peptide conjugate. The antioxidant and aromatic-cationic peptide can associate by any method known to those in the art. The following examples of peptide-antioxidant linkages are provided by way of illustration only, and are not intended to be limiting. In general, antioxidants can be linked to an aromatic-cationic peptide of the present disclosure by any suitable technique, with appropriate consideration of the need for pharmacokinetic stability and reduced overall toxicity to the subject. An antioxidant can be coupled to an aromatic-cationic peptide either directly or indirectly (e.g., via a linker group).

For covalent bond formation, a functional group on the antioxidant typically associates with a functional group on the aromatic-cationic peptide. For example, antioxidants may contain carboxyl functional groups, or hydroxyl functional groups. The free amine group of an aromatic-cationic peptide may be cross-linked directly to the carboxyl group of an antioxidant using 1-ethyl-3-[3-dimethylaminopropyl]carbodiimide hydrochloride (EDC or EDAC) or dicyclohexylcarbodiimide (DCC). Cross-linking agents can, for example, be obtained from Pierce Biotechnology, Inc., Rockford, Ill. The Pierce Biotechnology, Inc. website can provide assistance.

In some embodiments, a direct reaction between an additional active agent (e.g., an antioxidant) and an aromatic-cationic peptide (e.g., 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂ or a pharmaceutically acceptable salt thereof), is formed when each possesses a functional group capable of reacting with the other. Additionally or alternatively, a suitable chemical linker group can be used. A linker group can function as a spacer to distance the peptide and the antioxidant in order to avoid interference with, for example, binding capabilities. A linker group can also serve to increase the chemical reactivity of a substituent, and thus increase the coupling efficiency.

In some embodiments, an aromatic-cationic peptide as disclosed herein is coupled to more than one antioxidant. For example, in some embodiments, aromatic-cationic peptide is coupled to a mixture of at least two antioxidants. That is, more than one type of antioxidant can be coupled to one aromatic-cationic peptide. For instance, an antioxidant can be conjugated to an aromatic-cationic peptide to increase the effectiveness of the therapy, as well as lowering the required dosage necessary to obtain the desired therapeutic effect. Regardless of the particular embodiment, formulations with more than one moiety can be prepared in a variety of ways. For example, more than one moiety can be coupled directly to an aromatic-cationic peptide, or linkers that provide multiple sites for attachment (e.g., dendrimers) can be used. Alternatively, a carrier with the capacity to hold more than one antioxidant can be used.

Coupling between the aromatic-cationic peptide and the linker can be performed by any of the methods well-known in the art, including the use of carbodiimide coupling chemistry.

In some embodiments, the peptide conjugate is defined by any one of Formulas G, H, J, K, L, M, and N:

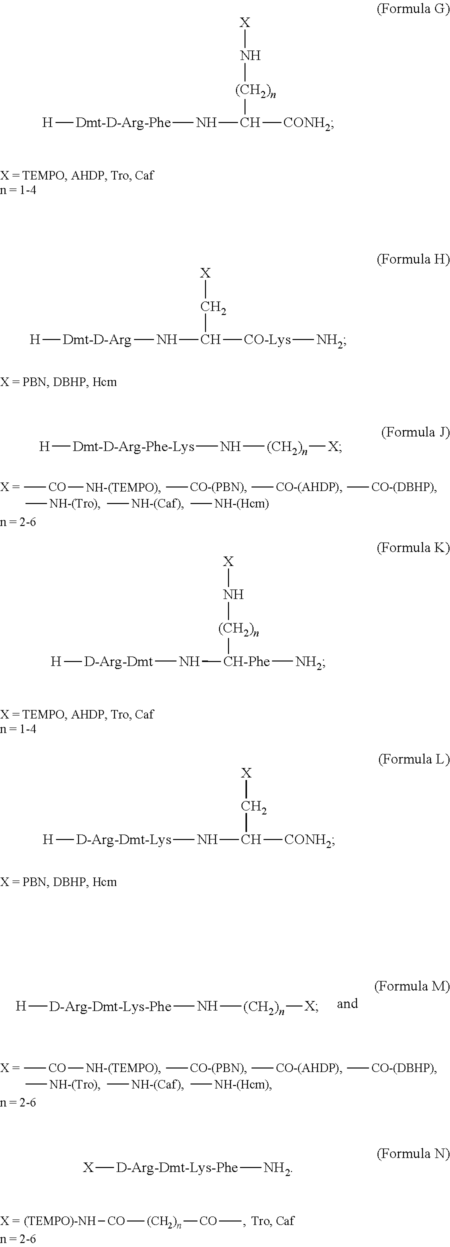

### VI. MODES OF ADMINISTRATION

Any method known to those in the art for contacting a cell, organ or tissue with compositions such as the peptide conjugates described herein (e.g., an aromatic-cationic peptide such as 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂, or pharmaceutically acceptable salt thereof, conjugated to an antioxidant selected from TEMPO, Trolox, PBN, AHDP, DBHP, Caf, and Hcm) may be employed. Suitable methods include in vitro, ex vivo, or in vivo methods.

In vitro methods typically include cultured samples. For example, a cell can be placed in a reservoir (e.g., tissue culture plate), and incubated with a compound under appropriate conditions suitable for obtaining the desired result. Suitable incubation conditions can be readily determined by those skilled in the art.

Ex vivo methods typically include cells, organs or tissues removed from a mammal, such as a human. The cells, organs or tissues can, for example, be incubated with the peptide conjugate under appropriate conditions. The contacted cells, organs or tissues are typically returned to the donor, placed in a recipient, or stored for future use. Thus, the compound is generally in a pharmaceutically acceptable carrier.

In vivo methods typically include the administration of a peptide conjugate, such as those described herein, to a mammal such as a human. When used in vivo for therapy, a peptide conjugate of the present technology are administered to a mammal in an amount effective in obtaining the desired result or treating the mammal. The effective amount is determined during pre-clinical trials and clinical trials by methods familiar to physicians and clinicians. The dose and dosage regimen will depend upon the degree of the infection in the subject, the characteristics of the particular peptide conjugate of the present technology used, e.g., its therapeutic index, the subject, and the subject's history.

An effective amount of a peptide conjugate of the present technology useful in the present methods, such as in a pharmaceutical composition or medicament, may be administered to a mammal in need thereof by any of a number of well-known methods for administering pharmaceutical compositions or medicaments. The peptide conjugate of the present technology may be administered systemically or locally.

The peptide conjugate of the present technology may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regimen). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide conjugate of the present technology contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′ dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine, and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic, and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic, and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, acetate, tartrate, trifluoroacetate, and the like.

The peptide conjugate of the present technology described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with the intended route of administration. Routes of administration include, for example, parenteral (e.g., intravenous, intradermal, intraperitoneal or subcutaneous), oral, respiratory (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J., USA) or phosphate buffered saline (PBS). In all cases, a composition for parenteral administration must be sterile and should be formulated for ease of syringeability. The composition should be stable under the conditions of manufacture and storage, and must be shielded from contamination by microorganisms such as bacteria and fungi.

In one embodiment, the peptide conjugate of the present technology is administered intravenously. For example, a peptide conjugate of the present technology may be administered via rapid intravenous bolus injection. In some embodiments, the peptide conjugate of the present technology is administered as a constant-rate intravenous infusion.

The peptide conjugate of the present technology may also be administered orally, topically, intranasally, intramuscularly, subcutaneously, or transdermally. In one embodiment, transdermal administration is by iontophoresis, in which the charged composition is delivered across the skin by an electric current.

Other routes of administration include intracerebroventricularly or intrathecally. Intracerebroventricularly refers to administration into the ventricular system of the brain. Intrathecally refers to administration into the space under the arachnoid membrane of the spinal cord. Thus, in some embodiments, intracerebroventricular or intrathecal administration is used for those diseases and conditions which affect the organs or tissues of the central nervous system.

The peptide conjugate of the present technology may also be administered to mammals by sustained release, as is known in the art. Sustained release administration is a method of drug delivery to achieve a certain level of the drug over a particular period of time. The level is typically measured by serum or plasma concentration. A description of methods for delivering a compound by controlled release can be found in international PCT Application No. WO 02/083106, which is incorporated herein by reference in its entirety.

Any formulation known in the art of pharmacy is suitable for administration of the peptide conjugate of the present technology. For oral administration, liquid or solid formulations may be used. Examples of formulations include tablets, gelatin capsules, pills, troches, elixirs, suspensions, syrups, wafers, chewing gum and the like. The peptide conjugates of the present technology can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

For systemic, intracerebroventricular, intrathecal, topical, intranasal, subcutaneous, or transdermal administration, formulations of the peptide conjugates of the present technology may utilize conventional diluents, carriers, or excipients etc., such as those known in the art to deliver the peptide conjugates of the present technology. For example, the formulations may comprise one or more of the following: a stabilizer, a surfactant, such as a nonionic surfactant, and optionally a salt and/or a buffering agent. Peptide conjugate of the present technology may be delivered in the form of an aqueous solution, or in a lyophilized form.

The stabilizer may comprise, for example, an amino acid, such as for instance, glycine; an oligosaccharide, such as, sucrose, tetralose, lactose; or a dextran. Alternatively, the stabilizer may comprise a sugar alcohol, such as, mannitol. In some embodiments, the stabilizer or combination of stabilizers constitutes from about 0.1% to about 10% weight for weight of the formulated composition.

In some embodiments, the surfactant is a nonionic surfactant, such as a polysorbate. Examples of suitable surfactants include Tween 20, Tween 80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example, sodium chloride, or sodium/potassium phosphate, respectively. In some embodiments, the buffering agent maintains the pH of the pharmaceutical composition in the range of about 5.5 to about 7.5. The salt and/or buffering agent is also useful to maintain the osmolality at a level suitable for administration to a human or an animal. In some embodiments, the salt or buffering agent is present at a roughly isotonic concentration of about 150 mM to about 300 mM.

Formulations of peptide conjugates of the present technology may additionally contain one or more conventional additives. Examples of such additives include a solubilizer such as, for example, glycerol; an antioxidant such as for example, benzalkonium chloride (a mixture of quaternary ammonium compounds, known as “quats”), benzyl alcohol, chloretone or chlorobutanol; an anesthetic agent such as for example a morphine derivative; and an isotonic agent etc., such as described herein. As a further precaution against oxidation or other spoilage, the pharmaceutical compositions may be stored under nitrogen gas in vials sealed with impermeable stoppers.

The mammal treated in accordance with the present technology may be any mammal, including, for example, farm animals, such as sheep, pigs, cows, and horses; pet animals, such as dogs and cats; and laboratory animals, such as rats, mice and rabbits. In one embodiment, the mammal is a human.

In some embodiments, peptide conjugates of the present technology are administered to a mammal in an amount effective in treating CRPS-I in the mammal. The effective amount is determined during pre-clinical trials and clinical trials by methods familiar to physicians and clinicians.

The peptide conjugate of the present technology may be administered systemically or locally. In one embodiment, the peptide conjugate of the present technology are administered intravenously. For example, the peptide conjugate of the present technology may be administered via rapid intravenous bolus injection. In one embodiment, the peptide conjugate of the present technology is administered as a constant-rate intravenous infusion.

The peptide conjugate of the present technology can be injected directly into a coronary artery during, for example, angioplasty or coronary bypass surgery, or applied onto coronary stents.

The peptide conjugate of the present technology may include a carrier, which can be a solvent or dispersion medium containing, for example, water, ethanol, polyol (for example, glycerol, propylene glycol, and liquid polyethylene glycol, and the like), or suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included in the composition to prevent oxidation. In many cases, it is desirable to include isotonic agents, for example, sugars, polyalcohols such as mannitol, sorbitol, or sodium chloride. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the peptide conjugate of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a peptide conjugate of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A peptide conjugate of the present technology can be formulated in a carrier system. The carrier can be a colloidal system. The colloidal system can be a liposome, a phospholipid bilayer vehicle. In one embodiment, the therapeutic peptide conjugate of the present technology is encapsulated in a liposome while maintaining protein integrity. As one skilled in the art will appreciate, there are a variety of methods to prepare liposomes. (See Lichtenberg, et al., Methods Biochem. Anal. 33:337-462 (1988); Anselem, et al., Liposome Technology, CRC Press (1993)). Liposomal formulations can delay clearance and increase cellular uptake (See Reddy, Ann. Pharmacother. 34 (78):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic peptide conjugate of the present technology can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother. 34:915-923 (2000). A polymer formulation for human growth hormone (hGH) has been used in clinical trials. (See Kozarich and Rich, Chemical Biology 2:548-552 (1998).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the peptide conjugates of the present technology are prepared with carriers that will protect the peptide conjugates of the present technology against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation (Mountain View, Calif., USA) and Nova Pharmaceuticals, Inc. (Sydney, AU). Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The peptide conjugate of the present technology can also be formulated to enhance intracellular delivery. For example, liposomal delivery systems are known in the art. See, e.g., Chonn and Cullis, Curr. Opin. Biotech. 6:698-708 (1995); Weiner, Immunometh. 4(3):201-9 (1994); Gregoriadis, Trends Biotechnol. 13(12):527-37 (1995). Mizguchi, et al., Cancer Lett. 100:63-69 (1996), describes the use of fusogenic liposomes to deliver a protein to cells both in vivo and in vitro

Dosage, toxicity and therapeutic efficacy of the peptide conjugate of the present technology can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the peptide conjugates of the present technology exhibit high therapeutic indices. While peptide conjugates of the present technology that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any peptide conjugate of the present technology used in the methods described herein, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the peptide conjugate of the present technology, sufficient for achieving a therapeutic or prophylactic effect, ranges from about 0.000001 mg per kilogram body weight per day to about 10,000 mg per kilogram body weight per day. In some embodiments, the dosage ranges will be from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of peptide conjugate of the present technology ranges from 0.1-10,000 micrograms per kg body weight. In one embodiment, peptide conjugate concentrations in a carrier range from 0.2 to 2000 micrograms per delivered milliliter. An exemplary treatment regimen entails administration once per day or once a week. Intervals can also be irregular as indicated by measuring blood levels of glucose or insulin in the subject and adjusting dosage or administration accordingly. In some methods, dosage is adjusted to achieve a desired fasting glucose or fasting insulin concentration. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of peptide conjugate of the present technology is defined as a concentration of the peptide conjugate of the present technology at the target tissue of 10⁻¹¹ to 10⁻⁶ molar, e.g., approximately 10⁻⁷ molar. This concentration may be delivered by systemic doses of 0.01 to 100 mg/kg or equivalent dose by body surface area. The schedule of doses is optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and the presence of other diseases. Moreover, treatment of a subject with a therapeutically effective amount of the therapeutic compositions described herein can include a single treatment or a series of treatments.

### Therapeutic Peptide Analogues

In some aspects, the present disclosure provides compositions including peptide conjugates of the present technology in combination with one or more active agents. In some embodiments, the active agents include any one or more of the aromatic-cationic peptides shown in Section II. In some embodiments, the aromatic-cationic peptide is 2′,6′-dimethyl-Tyr-D-Arg-Phe-Lys-NH₂, Phe-D-Arg-Phe-Lys-NH₂, or D-Arg-2′,6′-Dmt-Lys-Phe-NH₂.

In some embodiments, the aromatic-cationic peptides are modified so as to increase resistance to enzymatic degradation. One way of stabilizing peptides against enzymatic degradation is the replacement of an L-amino acid with a D-amino acid at the peptide bond undergoing cleavage. Peptide analogues are prepared containing one or more D-amino acid residues in addition to the D-Arg residue already present. Another way to prevent enzymatic degradation is N-methylation of the α-amino group at one or more amino acid residues of the peptides. This will prevent peptide bond cleavage by any peptidase. Examples include: H-D-Arg-Dmt-Lys(N^{α}Me)-Phe-NH₂; H-D-Arg-Dmt-Lys-Phe(NMe)-NH₂; H-D-Arg-Dmt-Lys(N^{α}Me)-Phe(NMe)-NH₂; and H-D-Arg(NaMₑ)-Dₘt(NMe)-L_{y}s(NaMₑ)-Phₑ(NMe)-NH₂. N′-methylated analogues have lower hydrogen bonding capacity and can be expected to have improved intestinal permeability. In some embodiments, the therapeutic peptide is modified by N-methylation of the α-amino group at one or more amino acid residues of the peptide.

An alternative way to stabilize a peptide amide bond (—CO—NH—) against enzymatic degradation is its replacement with a reduced amide bond (Ψ[CH₂—NH]). This can be achieved with a reductive alkylation reaction between a Boc-amino acid-aldehyde and the amino group of the N-terminal amino acid residue of the growing peptide chain in solid-phase peptide synthesis. The reduced peptide bond is predicted to result in improved cellular permeability because of reduced hydrogen-bonding capacity. Examples include: H-D-Arg-Ψ[CH₂—NH]Dmt-Lys-Phe-NH₂, H-D-Arg-Dmt-Ψ[CH₂—NH]Lys-Phe-NH₂, H-D-Arg-Dmt-LysΨ[CH₂—NH]Phe-NH₂, H-D-Arg-Dmt-Ψ[CH₂—NH]Lys-Ψ[CH₂—NH]Phe-NH₂, etc. In some embodiments, the therapeutic peptide is modified to include a reduced amide bond (Ψ[CH₂—NH]).

Stabilized peptide analogues may be screened for stability in plasma, simulated gastric fluid (SGF) and simulated intestinal fluid (SIF). An amount of peptide is added to 10 ml of SGF with pepsin (Cole-Palmer®, Vernon Hills, Ill.) or SIF with pancreatin (Cole-Palmer®, Vernon Hills, Ill.), mixed and incubated for 0, 30, 60, 90 and 120 min. The samples are analyzed by HPLC following solid-phase extraction. New analogues that are stable in both SGF and SIF are then be evaluated for their distribution across the Caco-2 monolayer. Analogues with apparent permeability coefficient determined to be >10⁻⁶ cm/s (predictable of good intestinal absorption) will then have their activity in reducing mitochondrial oxidative stress determined in cell cultures. Mitochondrial ROS is quantified by FACS using MitoSox for superoxide, and HyPer-mito (a genetically encoded fluorescent indicator targeted to mitochondria for sensing H₂O₂). Mitochondrial oxidative stressors can include t-butylhydroperoxide, antimycin and angiotensin. Therapeutic peptide analogues that satisfy all these criteria can then undergo large-scale synthesis.

It is predicted that the proposed strategies will produce a therapeutic peptide analog that would have oral bioavailability. The Caco-2 model is regarded as a good predictor of intestinal absorption by the drug industry.

### IX. EXAMPLES

The present technology is further illustrated by the following examples, which should not be construed as limiting in any way. For each of the examples below, any aromatic-cationic peptide described herein could be used. By way of example, but not by limitation, the aromatic-cationic peptide used in the examples below could be 2′6′-Dmt-D-Arg-Phe-Lys-NH₂, Phe-D-Arg-Phe-Lys-NH₂, or D-Arg-2′6′-Dmt-Lys-Phe-NH₂ or any one or more of the peptides shown in Section II and the antioxidant could be selected from TEMPO, Trolox, PBN, AHDP, DBHP, Caf, and Hcm.

### Example 1: Preparation of H-Dmt-D-Arg-Phe-Lys(NH-TEMPO)-NH₂

This example shows the production of Formula 0:

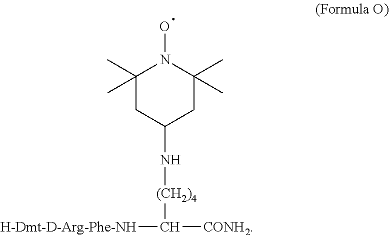

### Step 1: Synthesis of H-Lys(NH-TEMPO)-NH₂

Fmoc-Lys(NH-TEMPO)-NH₂ was synthesized using reaction conditions described in Shizuka et al., Bioorg. Med. Chem. Lett. (2007) 17, 1451-1454. 4-Oxo-TEMPO (1.59 mmol, 0.270 g) with acetic acid (1.91 mmol, 0.108 mL) in dry THF (4 mL) was added to Fmoc-Lys(NH₂×TFA)-NH₂ (1.59 mmol, 0.765 g) and TEA (1.6 mmol, 0.224 mL) in dry THF (10 mL). After 30 minutes, NaBH(OAc)₃ (3 eq., 4.77 mmol, 1.011 g) was added to the above mixture. The reaction was carried out in dry conditions (under Ar) overnight at room temperature. After completion of the reaction, saturated NaHCO₃ (20 mL) was added and the product was extracted with EtOAc (30 mL). The solution was dried over MgSO₄. After filtration and solvent evaporation, the product was obtained in the form of orange crystals (1.23 mmol, 0.645 g; yield 77.3%). Fmoc protection of the amino group was removed by treatment with 10% DEA/DMF (2 hours, room temperature). ¹H NMR (D₂O, 500 MHz, 6; ppm); 8.05 (2H, s), 7.81 (2H, s), 3.94-3.88 (1H, m), 3.71-3.63 (1H, m), 3.04-2.97 (2H, m), 2.90-2.88 (1H, s), 2.74-2.72 (1H, s), 2.38-2.31 (2H, m), 1.90-1.78 (4H, m), 1.67-1.58 (2H, m), 1.51-1.48 (1H, s), 1.38-1.35 (12H, m).

### Step 2: Synthesis of Fmoc-Dmt-D-Arg(Pbf)-Phe-OH

Fmoc-Dmt-D-Arg(Pbf)-Phe-OH was synthesized by a solid-phase technique using a chlorotrityl chloride resin, Fmoc protection of the α-amino group, Pbf protection of the D-Arg side chain, and HBTU/Cl-HOBt/DIPEA (1:1:2) as the coupling reagents. After cleavage from the resin with TFE/DCM (2:8), the product was obtained as a white powder (yield: 84%, ES/ML m/e=988).

### Step 3: Synthesis of H-Dmt-D-Arg-Phe-Lys(NH-TEMPO)-NH₂

Peptide bond formation between Fmoc-Dmt-D-Arg(Pbf)-Phe-OH (0.2 mmol, 0.197 g) and H-Lys(NH-TEMPO)-NH₂ (0.3 mmol, 0.0897 g) was performed in solution with DMF as solvent and HBTU/DIPEA (0.2 mmol, 0.076 g/0.5 mmol, 0.0875 mL) as the coupling reagents. The reaction was completed after 2 h. After DMF evaporation, the resulting oil was solidified by treatment with ethyl ether, affording the target compound as a white powder in quantitative yield (0.253 g, yield 100%). After Fmoc deprotection by 10% DEA/DMF treatment and Pbf side chain deprotection by HF/anisole treatment, the crude peptide conjugate was obtained as an off-white powder in quantitative yield (0.203 g, yield 100%). The peptide conjugate was purified by preparative reversed-phase HPLC using a gradient of 25-45% MeOH in 0.1% TFA/H₂O over a period 20 minutes at a flow rate of 12 mL/min. The pure peptide conjugate was obtained in the form of an off-white powder (ES/ML m/e=795).

### Example 2: Preparation of H-Dmt-D-Arg-Phe-Lys-NH—(CH²)₂—CO—NH-TEMPO

This example shows the production of Formula P:

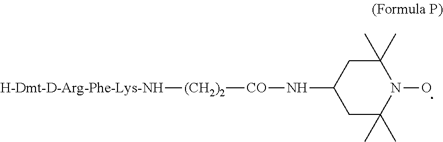

### Step 1: Synthesis of NH₂—(CH₂)₂—CO—NH-TEMPO

4-Amino-TEMPO (1 mmol, 0.171 g) in DMF (4 mL) was added to a stirring solution of Boc-β-Ala-OH (1 mmol, 0.189 g), HBTU (1 mmol, 0.379 g) and DIPEA (2.5 mmol, 0.435 mL) in DMF (15 mL). The reaction was complete after 15 minutes, as monitored by TLC. After evaporation of DMF, the product was purified by chromatography on a silica gel column, yielding Boc-β-Ala-TEMPO as an orange oil. Boc deprotection with 95% TFA in water was followed by solvent evaporation. Precipitation by addition of ethyl ether and lyophilization produced the TFA salt of NH₂—(CH₂)₂—CO—NH-TEMPO in the form of an orange oil (0.302 g, yield 84%). ¹H NMR (D₂O, 500 MHz, 6; ppm); 4.26-4.19 (1H, m), 3.26-3.00 (2H, t, J=6.5 Hz), 2.65-2.60 (2H, t, J=6.5 Hz), 2.20-2.04 (2H, d, J=14 Hz), 1.70-1.62 (2H, t, J=13 Hz), 1.41-1.37 (6H, s), 1.35-1.31 (6H, s).

### Step 2: Synthesis of Fmoc-Dmt-D-Arg(Pbf)-Phe-Lys(Boc)-OH

Fmoc-Dmt-D-Arg(Pbf)-Phe-Lys(Boc)-OH was synthesized by a solid-phase technique using a chlorotrityl chloride resin, Fmoc protection of the α-amino group, Pbf protection of the D-Arg side chain, Boc protection of the Lys side chain, and HBTU/Cl-HOB t/DIPEA (1:1:2) as the coupling reagents. After cleavage from the resin with TFE/DCM (2:8), the product was obtained in the form of a white foam (yield 85%, ES/ML m/e=1216).

### Step 3: Synthesis of H-Dmt-D-Arg-Phe-Lys-NH—(CH₂)₂—CO—NH-TEMPO

Coupling of Fmoc-Dmt-D-Arg(Pbf)-Phe-Lys(Boc)-OH and NH₂—(CH₂)₂—CO—NH-TEMPO was performed in solution. A 1.5-fold excess of the TFA salt of NH₂—(CH₂)₂—CO—NH-TEMPO (0.3 mmol, 0.107 g) and TEA (0.3 mmol, 0.042 mL) dissolved in DMF (5 mL) were added to a stirring solution of Fmoc-D-Arg(Pbf)-Phe-Lys(Boc)-OH (0.2 mmol, 0.243 g), Cl-HOBt (0.2 mmol, 0.034 g), HBTU (0.2 mmol, 0.076 g) and DIPEA (0.5 mmol, 0.0875 mL) in DMF (10 mL) The reaction was carried out for 15 hours and subsequent solvent evaporation yielded the protected peptide conjugate as a yellowish foam. Pbf- and Boc deprotection with TFA/H₂O/EDT (90:5:5) for 3 hours was followed by Fmoc deprotection with 10% DEA/DMF for 2 hours. Solvent evaporation and addition of Et₂O/hexane yielded the crude peptide conjugate in the form of an off-white powder (63% yield, 0.109 g). The peptide conjugate was purified by preparative reversed-phase HPLC using a gradient of 30-70% MeOH in 0.1% TFA/H₂O over a period of 40 minutes at a flow rate of 12 mL/min. The pure peptide conjugate was obtained in the form of an off-white powder (ES/ML m/e=865).

### Example 3: Preparation of H-Dmt-D-Arg-Phe-Lys(Tro[R or S])—NH₂

This example shows the production of Formula Q:

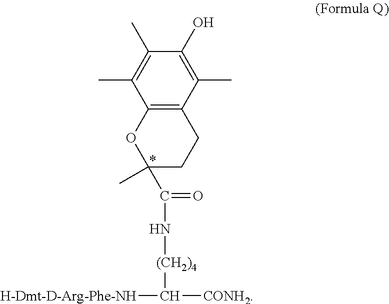

The peptides were synthesized by the solid-phase technique using a methylbenzylhydrylamine (MBHA) resin. Fmoc-Lys(Boc)-OH was attached to the resin using HBTU/DIPEA as the coupling reagents, and the Boc group was removed. Trolox (S isomer or R isomer; 4-fold excess) dissolved in DMF, was attached to the Lys side chain using the same coupling reagents described above. Using Fmoc α-amino group protection and Tos protection for the D-Arg side chain, the peptide was assembled using HBTU/DIPEA as the coupling reagents. Peptides were cleaved from the resin and completely deprotected by treatment with HF/anisole for 60 min at 0° C. Evaporation of HF was followed by washing of the resin with ethyl ether. Resin extraction with glacial acetic acid and lyophilization of the AcOH extracts gave the crude peptide conjugates in quantitative yield in solid form. Peptide conjugates were purified by preparative reversed-phase HPLC using a gradient of 20-45% MeOH in 0.1% TFA/H₂O over a period of 15 minutes, then a gradient of 45-55% over a period of 20 minutes at a flow rate of 12 mL/min. The pure peptide conjugates were obtained in the form of white powders (ES/ML m/e=872).

### Example 4: Preparation of H-Dmt-D-Arg-Phe-Lys(NH—CH₂-Tro)-NH₂ (R and S)

This example shows the production of Formula R:

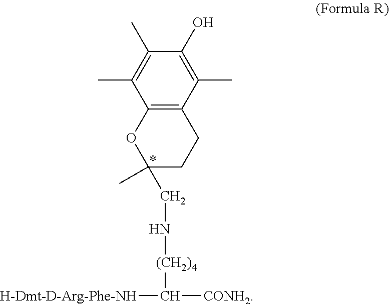

### Step 1: Synthesis of Trolox Aldehydes (R and S)

To Trolox (R or S) (2.1 mmol, 0.526 g), PyBOP (2.1 mmol, 1.089 g) and TEA (2.1 mmol, 0.294 mL) in DMF (15 mL), N,O-dimethylhydroxylamine hydrochloride (2.52 mmol, 0.246 g) and TEA (2.52 mmol, 0.353 mL) in DMF (10 mL) were added. After reaction over night, DMF was evaporated and the resulting oil was dissolved in AcOEt (30 mL). The solution was washed with brine (3×20 mL), dried over MgSO₄, filtered off and evaporated, yielding the crude N,O-dimethyl amides (Weinreb amides) of Trolox (R or S), respectively, in quantitative yield (2.1 mmol, 0.615 g). Reaction of the Weinreb amides of Trolox (R or S) with LiAlH₄ (3.36 mmol, 0.127 g) in THF (20 mL) under dry conditions (Argon atmosphere) gave the crude aldehydes of Trolox (R or S), respectively, in quantitative yield (2.1 mmol, 0.491 g).

### Step 2: Synthesis of H-Dmt-D-Arg-Phe-Lys(NH—CH₂-Tro)-NH₂ (R and S)

Both peptides were synthesized by a solid-phase technique using a MBHA resin, Fmoc protection of the α-amino group, Boc and Tos protection for the Lys and D-Arg side chains, respectively, and DIC/Cl-OBt as coupling reagents. After attachment of the protected Lys to the resin, its side chain protection was removed by treatment with 50% TFA/CH₂Cl₂ (v/v) and a reductive alkylation reaction was performed to form the reduced amide bond between the Trolox aldehyde and the ε-amino group of Lys. After subsequent completion of the peptide assembly, the peptides were cleaved from the resin by treatment with HF/anisole for 60 minutes at 0° C. Evaportion of the HF was followed by washing of the resin with ethyl ether. Resin extraction with glacial acetic acid and lyophilization of the AcOH extracts gave the crude peptide conjugates in quantitative yield in solid form. The compounds were purified by reversed-phase HPLC using a gradient of 20-55% MeOH in 0.15 TFA/H₂O over a period of 30 minutes at a flow rate of 12 mL/min. ES-ML m/e=858).

### Example 5: Preparation of H-Dmt-D-Ar₂-Phe-NH—(CH₂)—NH-Tro (R and S)

This example shows the production of Formula S:

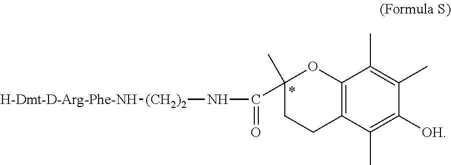

### Step 1: Synthesis of H₂N—(CH₂)₂—NH-Trolox (R and S)

To a solution of (R) or (S)-6-hydroxy-2,5,7,8-tetramethyl-chroman-2-carboxylic acid (1 mmol, 0.250 g) in DMF (6 mL) were added HBTU (1 mmol, 0.379 g) and DIPEA (2 mmol, 0.350 mL), followed by addition ofN-1-Boc-1,2-diaminoethane×HCl (1 mmol, 0.196 g) and triethylamine (1 mmol, 0.14 mL) in DMF (4 mL). After stirring the mixture for 2 h at RT, DMF was evaporated in vacuo. Ethylacetate and H₂O were added and the organic layer was washed twice with NaHCO₃ (sat.) and brine. The ethylacetate solution was dried over MgSO₄, filtered and evaporated to afford Boc-NH—(CH₂)₂—NH-Tro (R) (0.302 g, yield 77%) and Boc-NH—(CH₂)₂—NH-Tro (S) (0.382 g, yield 97%) as oils. The Boc group of the crude products was removed with TFA at 0° C. Evaporation of TFA and addition of ethyl ether resulted in the sedimentation of both products in the form of white crystals. TFA×H₂N—(CH₂)₂—NH-Tro (R): 0.283 g, yield 70%; TFA×H₂N—(CH₂)₂—NH-Tro (S): 0.384 g, yield 96%.

### Step 2: Synthesis of Fmoc-Dmt-D-Arg(Pbf)-Phe-OH

Fmoc-Dmt-D-Arg(Pbf)-Phe-OH was synthesized by a solid-phase technique using a 2-chlorotrityl resin, Fmoc protection of the α-amino group, Pbf protection of the D-Arg side chain and DIC/Cl-HOBt as coupling reagents. After cleavage from the resin with TFE/DCM (2:8), the product was obtained in the form of white crystals (yield: 85%, ES/ML m/e 858).

### Step 3: Synthesis of H-Dmt-D-Arg-Phe-NH—(CH₂)₂—NH-Tro (R and S)

Peptide bond formation between Fmoc-Dmt-D-Arg(Pbf)-Phe-OH (0.2 mmol, 0.197 g) and H₂N—(CH₂)₂-Tro (R or S) (0.2 mmol, 0.081 g) was performed in solution with DMF (10 mL) as solvent, with TEA (0.2 mmol, 0.028 mL) added, and HBTU//DIPEA (0.2 mmol, 0.197 g/0.5 mmol, 0.437 mL) in DMF (5 mL) as the coupling reagents. The reaction was competed after 2 h. After evaporation of the DMF, ethyl acetate and H₂O were added and the organic layer was washed with with NaHCO₃ (sat.) and brine. Drying of the AcOEt solution over MgSO₄, filtration and solvent evaporation yielded the crude protected peptide conjugates as white crystals (yield 77% for R- and S-compounds). Deprotection of the D-Arg(Pbf) side chain with TFA/H₂O/EDT (90:5:3) and subsequent removal of the Dmt Fmoc protection with 10% DEA/DMF yielded the crude peptides with yields of 77% (R) and 74% (S). The crude peptide conjugates were purified by reversed-phase HPLC using a gradient of 30-45% MeOH in 0.1% TFA/H₂O over a period of 10 minutes, followed by a gradient of 45-67% over 30 minutes at a flow rate of 12 mL/min (ES-ML m/e 787).

### Example 6: Preparation of H-D-Art-Dmt-Lys-Phe-NH—(CH₂)₂—CO—NH-TEMPO

This example shows the production of Formula T:

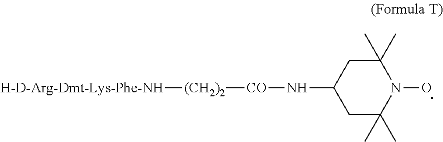

### Step 1: Synthesis of Fmoc-D-Arg(Pmc)-Dmt-Lys(Boc)-Phe-β-Ala-OH

Fmoc-D-Arg(Pmc)-Dmt-Lys(Boc)-Phe-β-Ala-OH was synthesized by a solid-phase technique using a H-β-Ala-2-chlorotrityl resin, Fmoc protection of the α-amino group, Boc and Pmc protection for the Lys and D-Arg side chains, respectively, and DIC/Cl-HOBt as the coupling reagents. Cleavage from the resin was performed by repetitive (10 times) 2-minute treatments with 1% TFA/DCM, followed by filtration into a flask containing a 10% pyridine/MeOH solution. Evaporation of the solvent down to 5% of the volume and treatment with ice-cold water afforded the protected peptide in quantitative yield and high purity (>95%). ES/ML m/e=1300.

### Step 2: Synthesis of H-D-Arg-Dmt-Lys-Phe-NH—(CH₂)₂—CO—NH-TEMPO

Amide bond formation between Fmoc-D-Arg(Pmc)-Dmt-Lys(Boc)-Phe-β-Ala-OH (0.25 mmol, 0.324 g) and 4-amino-TEMPO (0.375 mmol, 0.064 g) was performed in solution (DMF) using HBTU (0.25 mmol, 0.0947 g)/DIPEA (0.25 mmol, 0.217 mL) as the coupling reagents. After a reaction time of 15 hours and evaporation of DMF, the product was washed with EtOAc/H₂O/brine. The organic layer was dried over MgSO₄ and concentrated, providing the crude product as a yellow oil in quantitative yield. Deprotection of Fmoc (10% DEA/DMF, 2 hours) and Pmc and Boc (95% TFA/H₂O, 6 hours) yielded the crude peptide H-D-Arg-Dmt-Lys-Phe-NH—(CH₂)₂—CO—NH-TEMPO in the form of a yellowish powder (0.176 g, 81.5% yield). The peptide was purified by reversed-phase HPLC using a gradient of 20-30% MeOH in 0.1% TFA/H₂O over a period of 10 minutes, then a gradient of 30-40% MeOH in 0.1% TFA/H₂O over a period of 25 minutes, at a flow rate of 12 mL/min. ES/ML m/e=865.

### Example 7: Preparation of TEMPO-4-NH—CO—(CH₂)₂—CO-D-Art-Dmt-Lys-Phe-NH₂

This example shows the production of Formula U:

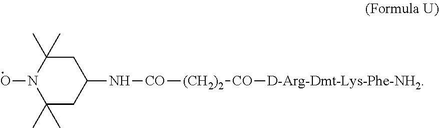

### Step 1: Synthesis of TEMPO-4-NH—CO—(CH₂)₂—CO₂H

4-Amino-TEMPO (3 mmol, 0.513 g) dissolved in DMF (6 mL) was added to a mixture of mono-ethyl succinate (3 mmol, 0.426 mL), HBTU (3 mmol, 1.137 g) and DIPEA (7.5 mmol, 1.035 mL) dissolved in DMF (8 mL). After completion of the reaction (2 hours), DMF was evaporated in vacuo and the resulting oil was dissolved in a mixture of EtOAc (25 mL) and H₂O (20 mL). The organic layer was washed with saturated NaHCO₃ (3×10 mL) and brine (3×10 mL), dried over MgSO₄, filtered, and evaporated, furnishing the crude product TEMPO-4-NH—CO—(CH₂)₂—CO₂Et as an orange oil in quantitative yield. The product was dissolved in MeOH (20 mL) and 1N NaOH (6 mL) was added dropwise. After completion of the reaction (30 minutes), MeOH was evaporated and the resulting oily crystals were dissolved in a mixture of EtOAc (20 mL) and H₂O (20 mL). The oily crystals were subjected to acidification with 1N HCl to pH 3, followed by separation of the organic and aqueous layers, drying of the organic layers over MgSO₄, filtration, and solvent evaporation in vacuo, yielding the target product TEMPO-4-NH—CO—(CH₂)₂—COOH in the form of orange crystals (1.8 mmol, 0.488 g, yield=60%).

### Step 2: Synthesis of TEMPO-4-NH—CO—(CH₂)₂—CO-D-Arg-Dmt-Lys-Phe-NH₂

TEMPO-4-NH—CO—(CH₂)₂—CO-D-Arg-Dmt-Lys-Phe-NH₂ was prepared by the solid-phase technique using a MBHA resin, Fmoc α-amino protection, Boc and Pbf protection of the side chains of Lys and D-Arg, respectively, and DIC/Cl-HOBt as the coupling reagents. After assembly of the tetrapeptide, Fmoc protection of the N-terminal D-Arg residue was removed and amide bond formation between TEMPO-4-NH—CO—(CH₂)₂—COOH and the resin-bound tetrapeptide was performed using DIC/Cl-HOBt as the coupling c reagents. The peptide conjugate was cleaved from the resin and deprotected by HF/anisole treatment and after lyophilization was obtained in quantitative yield. The crude product was purified by preparative reversed-phase HPLC using a gradient of 20-30 MeOH in 0.1% TFA/H₂O over a period of 20 minutes at a flow rate of 12 mL/min. ES/ML m/e=893.

### Example 8: Preparation of H-D-Arg-Dmt-Lys(TEMPO)-Phe-NH₂

This example shows the production of Formula V:

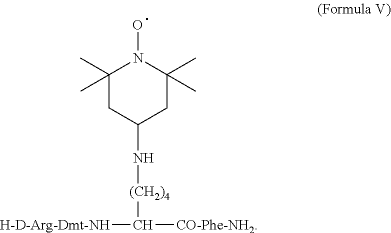

The peptide was synthesized by the solid-phase method using a p-methylbenzhydrylamine resin, Fmoc α-amino group protection, Boc- and Pbf-protection for the side chains of Lys and D-Arg, respectively, and Cl-HOBt/DIC as the coupling reagents. After assembly of the resin-bound C-terminal dipeptide segment, the Boc group on the Lys residue was removed. The resin was then dried and transferred to a two-neck round bottom flask. The resin was allowed to react with 4-oxo-TEMPO (4 eq.) and acetic acid (6 eq.) in dry THF for 1 hour prior to the addition of NaBH(OAc)₃ (12 eq.). Reductive amination was carried out for 15 hours. After completion of the reaction, the mixture was transferred to the solid-phase synthesis reaction vessel and the resin was washed thoroughly with DMF, isopropanol and DCM. The assembly of the tetrapeptide was completed by attaching the two N-terminal residues. After cleavage from the resin with HF/anisole (60 minutes at 0° C.) the crude peptide conjugate was obtained in solid form with a yield of 80% and was purified by reversed-phase HPLC using a gradient of 30-50% MeOH in 0.1% TFA/H₂O over a period of 25 minutes at a flow rate of 12 mL/min. ES/ML m/e=794.

### Example 9: In Vitro Opioid Activity Profiles

This example shows the in vitro opioid activity of the peptide conjugates. Peptide conjugates of Table 5 were tested in functional assays based on inhibition of electrically evoked contractions of the guinea pig ileum (GPI) and the mouse vas deferens (MVD). The GPI assay is representative for μ opioid receptor (MOR) interactions, whereas in the MVD assay opioid effects are primarily mediated by the δ opioid receptor (DOR). The assays were carried out as described in DiMaio et al., J. Med. Chem. (1982) 25, 1432-1438. Binding affinities for μ and δ receptors were determined by displacing, respectively, [³H]DAMGO and [³H]DSLET from rat brain membrane binding sites (see Schiller et al., Biochem. Biophys. Res. Commun. (1978) 85, 1332-1338). The in vitro opioid activity profiles of select compounds are presented in Table 5.

TABLE 5

| | GPI | MVD | ReceptorBinding | |
|---|---|---|---|---|
| | IC₅₀ | IC₅₀ | Kᵢ^{μ} | Kᵢ^{δ} |
| Compound | (nM) | (nM) | (nM) | (nM) |
| H-Dmt-D-Arg-Phe-Lys | 3.83 | 1.15 | 0.263 | 30.4 |
| (NH-TEMPO)-NH₂ | | | | |
| H-Dmt-D-Arg-Phe-Lys-NH-(CH₂)₂- | 445 | 262 | 4.56 | 2.15 |
| CO-NH-TEMPO | | | | |
| H-Dmt-D-Arg-Phe-Lys(Tro[R])-NH₂ | 5.06 | 0.847 | 0.273 | 3.64 |
| H-Dmt-D-Arg-Phe-Lys(Tro[S]-NH₂ | 2.56 | 3.76 | 0.252 | 3.48 |
| H-Dmt-D-Arg-Phe-Lys-NH₂ | 1.41 | 23.1 | 0.143 | 2100 |
| ([Dmt¹]DALDA) | | | | |

In comparison with the [Dmt¹]DALDA parent (H-Dmt-D-Arg-Phe-Lys-NH₂), three of the four analogues showed comparable μ and δ receptor binding affinities in the subnanomolar range, as well as preference for μ over δ receptors. The very high μ receptor binding affinities of these compounds are in agreement with their high μ opioid agonist potencies determined in the functional GPI assay (IC₅₀s in the low nanomolar range).

### Example 10: Evaluation of H-Dmt-D-Arg-Phe-Lys(NH-TEMPO)-NH₂ in an Animal Model of Complex Regional Pain Syndrome-Type I (CRPS-I)

This example demonstrates the in vivo efficacy of the peptide conjugates described herein in treating complex regional pain syndrome-Type I.

H-Dmt-D-Arg-Phe-Lys(NH-TEMPO)-NH₂ was tested in a chronic post-ischemia pain (CPIP) rat model (see Coderre et al., Pain (2004) 112, 94-105) of CRPS-I in comparison with [Dmt¹]DALDA and morphine (subcutaneous (s.c.) administration). The analgesic potencies (ED₅₀ values) of the compounds were determined based on their ability to reverse mechanical allodynia in CPIP rats (Table 6).

TABLE 6

| | ED₅₀ | Potency |
|---|---|---|
| Compound | (mg/kg) ± SEM | Ratio |
| H-Dmt-D-Arg-Phe-Lys(NH-TEMPO)-NH₂ | 0.0228 ± 0.0096 | 67.8 |
| H-Dmt-D-Arg-Phe-Lys-NH₂([Dmt¹]DALDA) | 0.103 ± 0.046 | 15.0 |
| morphine | 1.546 ± 0.664 | 1 |

H-Dmt-D-Arg-Phe-Lys(NH-TEMPO)-NH₂ was observed to be 67.8-fold more potent than morphine and 4.5-fold more potent than [Dmt¹]DALDA. Since the compound was given s.c., this result indicates that the compound was capable of crossing the blood-brain barrier to produce a centrally mediated analgesic effect. This result show a synergistic effect of the peptide conjugate including [Dmt¹]DALDA and TEMPO. Additionally, this result indicates that [Dmt¹]DALDA analogues conjugated to TEMPO have therapeutic potential for treatment of CRPS-I.

These results show that H-Dmt-D-Arg-Phe-Lys(NH-TEMPO)-NH₂ is useful in treating CRPS in a CPIP rat model. These results show that the peptide conjugates described herein are useful for the treatment of complex regional pain syndrome.

### Example 11: Evaluation of Antioxidant Activity of H-Dmt-D-Arg-Phe-Lys-NH₂ ([Dmt¹]DALDA), H-Dmt-D-Arg-Phe-Lys(Tro[S])—NH₂, H-Dmt-D-Arg-Phe-Lys(NH—CH₂-Tro)-NH₂ (S), and H-Dmt-D-Arg-Phe-Lys(NH-TEMPO)-NH₂

This example demonstrates that peptide conjugates of the present technology have increased antioxidant activity as compared to the antioxidant activity of the aromatic-cationic peptide in the peptide conjugate alone.

Antioxidant activities of peptides were determined by reduction of linoleic acid peroxidation initiated with 2,2′-azabis(2-amidinopropane) (ABAP), as described in Pryor et al., J. Org. Chem., 58: 3521-3535 (1993). A constant rate of linoleic acid peroxidation was reached 20 minutes after the addition of ABAP to the cuvette (dashed line). An aromatic-cationic peptide (H-Dmt-D-Arg-Phe-Lys-NH₂ ([Dmt¹]DALDA) (●)) and peptide conjugates containing the above aromatic-cationic peptide (H-Dmt-D-Arg-Phe-Lys(Tro[S])—NH₂ (▪), H-Dmt-D-Arg-Phe-Lys(NH—CH₂-Tro)-NH₂ (S) (▴), H-Dmt-D-Arg-Phe-Lys(NH-TEMPO)-NH₂ (□)) were added after constant rate of linoleic acid peroxidation was established. Formation of conjugated dienes was measured spectrophotometrically at 234 nm and the reduction of the peroxidation rate after addition of peptides was determined.

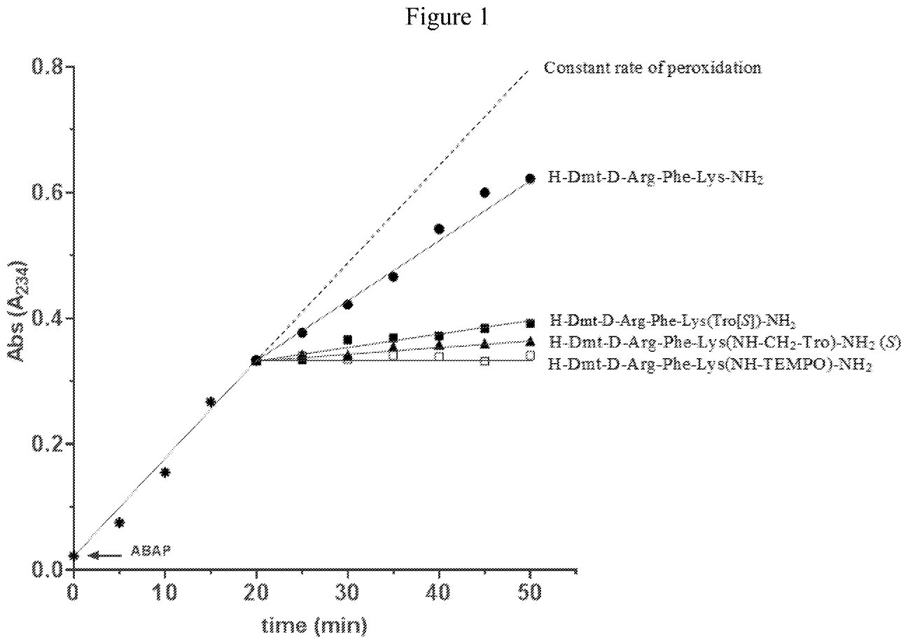

FIG. 1 shows that peptide conjugates (H-Dmt-D-Arg-Phe-Lys(NH-TEMPO)-NH₂, H-Dmt-D-Arg-Phe-Lys(NH—CH₂-Tro)-NH₂ (S) and H-Dmt-D-Arg-Phe-Lys(TroN)—NH₂) showed a greater reduction in the rate of peroxidation cause by ABAP as compared to H-Dmt-D-Arg-Phe-Lys-NH₂ ([Dmt¹]DALDA).

These results show that H-Dmt-D-Arg-Phe-Lys(NH-TEMPO)-NH₂, H-Dmt-D-Arg-Phe-Lys(NH—CH₂-Tro)-NH₂ (S) and H-Dmt-D-Arg-Phe-Lys(Tro[S])-NH₂ have greater antioxidant activity as compared to H-Dmt-D-Arg-Phe-Lys-NH₂. As such, the peptide conjugates of the present technology have greater antioxidant activity as compared to the antioxidant activity of the aromatic-cationic peptide in the peptide conjugate alone.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

What is claimed is:

1. A composition comprising an antioxidant directly or indirectly conjugated to an aromatic-cationic peptide, wherein the aromatic-cationic peptide is selected from the group consisting of 2′,6′-Dmt-D-Arg-Phe-Lys-NH₂, Phe-D-Arg-Phe-Lys-NH₂, D-Arg-2′,6′-Dmt-Lys-Phe-NH₂, and a peptide of Tables A-E, and wherein the antioxidant is selected from the group consisting of TEMPO, Tro, PBN, AHDP, DBHP, Caf, and Hcm.

2. The composition of claim 1, wherein the antioxidant is directly or indirectly conjugated to the N-terminus or C-terminus of the aromatic-cationic peptide.

3. The composition of claim 1, wherein the antioxidant is directly or indirectly conjugated to a sidechain of an amino acid residue of the aromatic-cationic peptide.

4. The composition of claim 1, wherein the antioxidant is covalently bound to the aromatic-cationic peptide through a nitrogen or oxygen atom on the aromatic-cationic peptide.

5. The composition of any one of claims 1-3, wherein the antioxidant is indirectly conjugated to the aromatic-cationic peptide through a linker.

6. The composition of claim 5, wherein the linker is covalently bound to the aromatic-cationic peptide through a nitrogen on the aromatic-cationic peptide.

7. The composition of any one of claim 1-3, 5 or 6 wherein the antioxidant is indirectly conjugated to the aromatic-cationic peptide through a C₁-C₁₂ linker.

8. The composition of any one of claims 5-7, wherein the linker comprises one or more groups independently selected from the group consisting of a carbonyl, an amine, and an alkylene group.

9. The composition of any one of claims 5-8, wherein the linker is selected from the group consisting of —C(O)—(C₁-C₆ alkylene)-C(O)—, —C(O)—(C₁-C₆ alkylene)-NH—, and —NH—(C₁-C₆ alkylene)-NH—.

10. The composition of any one of claims 1-9, wherein the aromatic-cationic peptide is selected from the group consisting of 2′,6′-Dmt-D-Arg-Phe-Lys-NH₂, Phe-D-Arg-Phe-Lys-NH₂, and D-Arg-2′,6′-Dmt-Lys-Phe-NH₂.

11. The composition of any one of claims 1-10, wherein the aromatic-cationic peptide comprises H-2′,6′-Dmt-D-Arg-Phe-Lys-NH₂.

12. The composition of any one of claims 1-10, wherein the aromatic-cationic peptide comprises H-D-Arg-2′,6′-Dmt-Lys-Phe-NH₂.

13. The composition of claim 1, wherein the peptide conjugate has a structure of
Formula G, wherein X=TEMPO, AHDP, Tro, or Caf, and n=1-4;
Formula H, wherein X=PBN, DBHP, or Hcm;
Formula J, wherein X=—CO—NH-(TEMPO), —CO—(PBN), —CO-(AHDP), —CO-(DBHP), —NH-(Tro), —NH—(Caf), or —NH—(Hcm), and n=2-6;
Formula K, wherein X=TEMPO, AHDP, Tro or Caf, and n=1-4;
Formula L, wherein X=PBN, DBHP, or Hcm;
Formula M, wherein X=—CO—NH-(TEMPO), —CO—(PBN), —CO-(AHDP), —CO-(DBHP), —NH-(Tro), —NH—(Caf), or —NH—(Hcm), and n=2-6; or
Formula N, wherein X=(TEMPO)-NH—CO—(CH₂)ₙ—CO—, Tro or Caf, and n=2-6.

14. A method for treating, ameliorating or preventing complex regional pain syndrome in a subject in need thereof, comprising administering a therapeutically effective amount of a peptide conjugate of any one of claims 1-13 to the subject thereby treating, ameliorating, or preventing complex regional pain syndrome.

15. The method of claim 14, wherein the medical disease or condition is characterized by mitochondrial permeability transition.

16. The method of claim 14, wherein complex regional pain syndrome is complex regional pain syndrome-Type I (CRPS-I).

17. A composition comprising an aromatic-cationic peptide disclosed in Section II directly or indirectly conjugated to an antioxidant selected from TEMPO, PBN, AHDP, DBHP, Tro, Caf, and Hcm.

18. The composition of claim 17, wherein the aromatic-cationic peptide is selected from 2′,6′-Dmt-D-Arg-Phe-Lys-NH₂, Phe-D-Arg-Phe-Lys-NH₂, and D-Arg-2′,6′-Dmt-Lys-Phe-NH₂.

19. The composition of claim 17, wherein the aromatic-cationic peptide comprises H-2′,6′-Dmt-D-Arg-Phe-Lys-NH₂.

20. The composition of claim 17, wherein the aromatic-cationic peptide comprises H-D-Arg-2′,6′-Dmt-Lys-Phe-NH₂.

21. A method for treating or preventing complex regional pain syndrome, comprising administering a therapeutically effective amount of a composition of any one of claims 17-20.

22. The method of claim 21, wherein complex regional pain syndrome is complex regional pain syndrome-Type I (CRPS-I).
